(19) United States
(12) Patent Application Publication
(10) Pub. No.: US 2026/0272314 A1
(43) Pub. Date: Sep. 17, 2026

(54) BLOOD PRESSURE PULSE WAVE EXAMINATION SYSTEM

(71) Applicant: FUKUDA DENSHI CO., LTD., Bunkyo-ku, Tokyo (JP)

(72) Inventors: Kensaku Yoshino, Tokyo (JP); Ayaka Gonda, Tokyo (JP); Tomoyuki Yamamoto, Tokyo (JP)

(21) Appl. No.: 19/469,405

(22) PCT Filed: Mar. 28, 2024
(86) PCT No.: PCT/JP2024/012803

§ 371 Date: Sep. 26, 2025

(30) Foreign Application Priority Data
Mar. 31, 2023 (JP) 2023-058496
Mar. 31, 2023 (JP) 2023-058512
Mar. 31, 2023 (JP) 2023-058517
Mar. 31, 2023 (JP) 2023-058525
Mar. 31, 2023 (JP) 2023-058529

(51) Int. Cl. A61B 5/021; A61B 5/00; A61B 5/022

(52) U.S. Cl. CPC A61B 5/02125; A61B 5/0004; A61B 5/002; A61B 5/02233

(57) ABSTRACT

Provided is a blood pressure pulse wave examination system that can be configured so as to be hoseless. A blood pressure pulse wave examination system comprising: a plurality of wireless cuff devices that are mounted at prescribed sites on a subject; and a blood pressure pulse wave examination device that performs wireless communication with the plurality of wireless cuff devices, controls the plurality of wireless cuff devices, collects pulse wave data from the plurality of wireless cuff devices, and examines the blood vessel state of the subject. Each of the plurality of wireless cuff devices has a cuff that is wound around a prescribed site on the subject, and a wireless unit that is attached to the cuff.

### TECHNICAL FIELD

The present disclosure relates to a blood pressure pulse wave analyzing system.

### BACKGROUND ART

In the related art, as an apparatus that examines a state of a blood vessel such as an arterial stiffness, there is a blood pressure pulse wave analyzing apparatus (see, for example, PTL 1). The blood pressure pulse wave analyzing apparatus acquires blood pressure pulse waves of a subject with a plurality of cuffs mounted on limbs or toes of the subject. The blood pressure pulse wave analyzing apparatus obtains an indicator of arterial stiffness, such as a pulse wave velocity (PWV) or an ankle brachial index (ABI), based on the blood pressure pulse waves obtained by the plurality of cuffs.

The blood pressure pulse wave analyzing apparatus and the plurality of cuffs are connected by hoses. The blood pressure pulse wave analyzing apparatus is provided with an air pump and a pressure sensor, and air output from the air pump is supplied to each cuff via the hose.

### CITATION LIST

### Patent Literature

### PTL 1

Japanese Patent Application Laid-Open No. 2016-158943

### Non-Patent Literature

### NPL 1

ANALYSIS STUDY OF TIME SYNCHRONIZATION PROTOCOLS IN WIRELESS SENSOR NETWORKS, Salim el Khediri, Laboratory of Electrical Engineering and Information Technology 2012

### NPL 2

H. Kopetz and W. Schwabl. Global time in distributed real-time systems, Technical Report 15/89, Technische Universität Wien, 1989.

### SUMMARY OF INVENTION

### Technical Problem

As described above, in the related art, hoses are necessary for connecting the blood pressure pulse wave analyzing apparatus and the plurality of cuffs. However, the hoses also come into contact with the subject, which is unhygienic. In addition, there is a danger that a foot may be caught in a hose or the like. Further, it takes time to prepare the hoses or to untangle the hoses, which means that the time required to perform the examination is increased.

Therefore, it is desirable to develop a hoseless configuration. When the hoseless configuration is achieved, it is considered that the above-described problems can be solved and the bed area where the examination is performed can be simplified, making the examination easier.

However, in the related art, the hoseless configuration in the blood pressure pulse wave examination has not been sufficiently studied.

One object of the present disclosure is made in consideration of the above-described points, and is to provide a blood pressure pulse wave analyzing system that can achieve a hoseless configuration. In addition, the present disclosure provides various technical ideas for achieving the hoseless configuration.

### Solution to Problem

One aspect of a blood pressure pulse wave analyzing system according to the present disclosure includes the following:
a plurality of wireless cuff devices each to be mounted on a predetermined site of a subject; and
a blood pressure pulse wave analyzing apparatus that performs wireless communication with the plurality of wireless cuff devices, controls the plurality of wireless cuff devices, and collects pulse wave data from the plurality of wireless cuff devices to examine a blood vessel state of the subject, in which
each of the plurality of wireless cuff devices includes
   a cuff to be wound around the predetermined site of the subject, and
   a wireless communication unit attached to the cuff.

### Advantageous Effects of Invention

The present invention is capable of realizing a blood pressure pulse wave analyzing system that can achieve a hoseless configuration.

### BRIEF DESCRIPTION OF DRAWINGS

FIG. 1 is a perspective view illustrating a configuration of a wireless cuff device of Embodiment 1;

FIG. 2 is a perspective view illustrating a plurality of wireless cuff devices used for a blood pressure pulse wave examination of Embodiment 1;

FIG. 3 illustrates a state in which the wireless cuff devices are mounted on a subject;

FIG. 4 is a block diagram illustrating a configuration of a wireless communication unit of Embodiment 1;

FIG. 5 is a block diagram illustrating a configuration of a blood pressure pulse wave analyzing apparatus of Embodiment 1;

FIG. 6 is a block diagram illustrating a configuration of a blood pressure pulse wave analyzing apparatus according to another form 1 of Embodiment 1;

FIG. 7 is a diagram for describing estimation of a blood vessel length by a blood vessel length estimator);

FIG. 8 is a diagram for describing estimation of a blood vessel length by a blood vessel length estimator;

FIG. 9 is a block diagram illustrating a configuration of a blood pressure pulse wave analyzing apparatus according to another form 3 of Embodiment 1;

FIG. 10 is a perspective view illustrating a configuration of a wireless cuff device of Embodiment 2;

FIG. 11 is a perspective view illustrating a plurality of wireless cuff devices used for a blood pressure pulse wave examination of Embodiment 2;

FIG. 12 is a block diagram illustrating a configuration of a wireless communication unit of Embodiment 2;

FIGS. 13A to 13C illustrate a format example of a packet transmitted from a wireless communication unit of Embodiment 3 to the blood pressure pulse wave analyzing apparatus, in which FIG. 13A illustrates a data configuration of the packet, FIG. 13B illustrates the content of the packet transmitted from the wireless communication unit to the blood pressure pulse wave analyzing apparatus before the transmission of measurement data, and FIG. 13C illustrates the content of the packet when the measurement data is transmitted after the packet illustrated in FIG. 13B is transmitted;

FIG. 14 is a diagram for describing factors of the delay time of a message from a wireless transmitter to a wireless receiver;

FIG. 15 is a diagram for describing time synchronization when a successive approximation register type analog-digital converter (hereinafter referred to as “ADC”) is used;

FIG. 16 is a diagram for describing time synchronization when a delta sigma (ΔΣ) type ADC is used;

FIG. 17 illustrates a state in which a time synchronization packet is lost;

FIG. 18 is a block diagram for describing a time synchronization method according to Embodiment 3;

FIG. 19 illustrates initial transmission from a host device to a client device;

FIG. 20 illustrates transmission from the host device to the client device up to the second time;

FIG. 21 illustrates transmission from the host device to the client device up to the third time;

FIG. 22 is a diagram for describing setting of a start point of a read pointer based on a Td value;

FIG. 23 is a diagram for describing setting of an end point of a read pointer based on a Td value;

FIG. 24 illustrates a state of timing deviation in the client device;

FIG. 25 illustrates a processing example in which the timing deviation is prevented according to Embodiment 3;

FIG. 26 is a block diagram illustrating a configuration example for performing the processing of FIG. 25;

FIG. 27 is a diagram for describing processing when radio wave loss (packet loss) occurs;

FIG. 28 illustrates an example in which the synchronization method according to Embodiment 3 is applied to a system including a plurality of client devices;

FIG. 29 illustrates an example in which an ADC clock lags behind a reference clock;

FIG. 30 illustrates an example in which the ADC clock advances faster than the reference clock;

FIG. 31 illustrates an operation example of an algorithm of Bresenham;

FIG. 32 illustrates an operation example of a modified Bresenham's algorithm according to Embodiment 3;

FIG. 33 illustrates a modification example of the algorithm of Bresenham; and

FIG. 34 illustrates an example in which the time synchronization method and a wireless system according to Embodiment 3 are applied to a medical apparatus.

### DESCRIPTION OF EMBODIMENTS

### 1> Embodiment 1

### <1-1> Knowledge Leading to Embodiment 1

Before describing Embodiment 1, knowledge of the inventors of the present disclosure leading to Embodiment 1 will be described. The inventors of the present disclosure conceived of achieving a hoseless configuration by wirelessly connecting a cuff with a blood pressure pulse wave analyzing apparatus.

The inventors have studied connection between a cuff and a blood pressure pulse wave analyzing apparatus in the related art in order to achieve the hoseless configuration. A hose is led out from each of the cuffs to be mounted on a left upper arm, a right upper arm, a left lower limb, and a right lower limb. The blood pressure pulse wave analyzing apparatus is provided with four connectors for connecting these four hoses. Here, the hose led out from the cuff for the left upper arm is connected to the connector for the left upper arm, the hose led out from the cuff for the right upper arm is connected to the connector for the right upper arm, the hose led out from the cuff for the left lower limb is connected to the connector for the left lower limb, and the hose led out from the cuff for the right lower limb is connected to the connector for the right lower limb.

For example, when incorrect connection is performed, such as the hose led out from the cuff for the left upper arm being connected to the connector for the left lower limb, an incorrect examination result is obtained in the blood pressure pulse wave analyzing apparatus. Therefore, labels of different colors are respectively attached to the hoses of the cuffs, and labels of corresponding different colors are respectively attached to the connectors. For example, yellow labels are attached to the hose led out from the cuff for the left upper arm and the connector for the left upper arm, and red labels are attached to the hose led out from the cuff for the right upper arm and the connector for the right upper arm. In this manner, it is possible to prompt a medical worker to connect the hose with the connector of the same color, thereby preventing incorrect connection between the hose and the connector.

However, when a plurality of cuffs are wirelessly connected to the blood pressure pulse wave analyzing apparatus, the hoses are also become unnecessary, so that it is not possible to associate which cuff is for which mounting site via connection of the hose to the connector. Therefore, a certain means for recognizing which cuff is for which mounting site is necessary in the blood pressure pulse wave analyzing apparatus.

The inventors of the present disclosure have found a configuration suitable for the blood pressure pulse wave analyzing apparatus to recognize which cuff is for which mounting site even when a hoseless configuration is achieved, and have arrived at the present disclosure.

### <1-2> Configuration of Embodiment 1

Hereinafter, Embodiment 1 of the present disclosure will be described in detail with reference to the drawings.

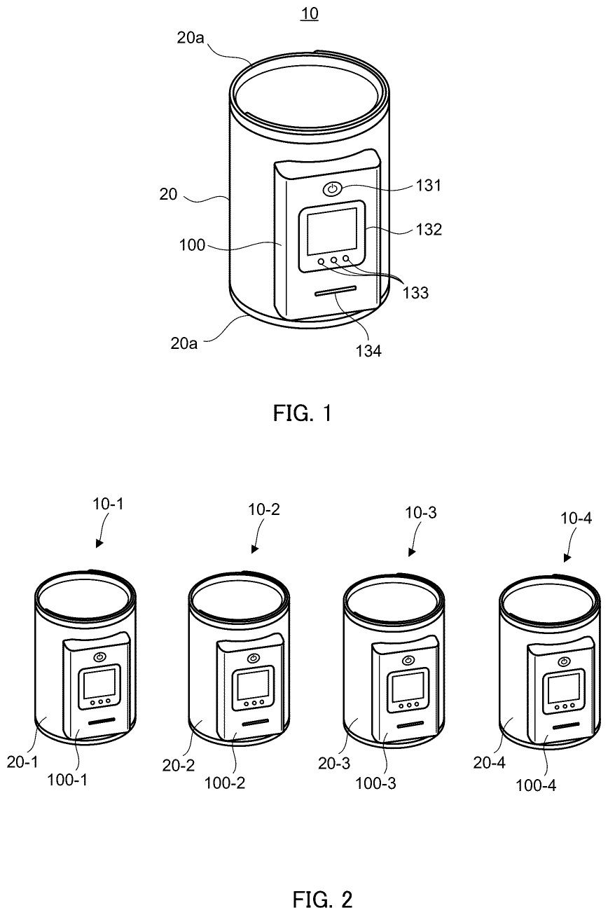

FIG. 1 is a perspective view illustrating a configuration of a wireless cuff device of Embodiment 1.

Wireless cuff device 10 includes cuff 20 and wireless communication unit 100.

Cuff 20 includes an air bag (not illustrated) in the inside thereof and is wound around a predetermined site of a subject. Edge 20a of each cuff 20 is colored according to the part of the subject to which cuff 20 is to be mounted. For example, edge 20a of cuff 20 for a left upper arm is yellow, edge 20a of cuff 20 for a right upper arm is red, edge 20a of cuff 20 for a left lower limb is green, and edge 20a of cuff 20 for a right lower limb is black. Furthermore, each cuff 20 has written thereon a character (not illustrated) indicating the mounting site for the cuff. A medical worker attaches each cuff 20 on a part corresponding to the color and the character of the cuff 20. As cuff 20, a known configuration that has been used in the related art for measuring a blood pressure pulse wave can be used, and thus the description thereof will be omitted herein.

Wireless communication unit 100 is detachably attached to cuff 20. A structure for attaching wireless communication unit 100 to cuff 20 can be achieved, for example, by forming rails that can be engaged with each other on the front surface of cuff 20 and the rear surface of wireless communication unit 100. In this manner, wireless communication unit 100 can be easily attached to and detached from cuff 10 by sliding wireless communication unit 100 with respect to cuff 20. Of course, the attachment structure is not limited thereto.

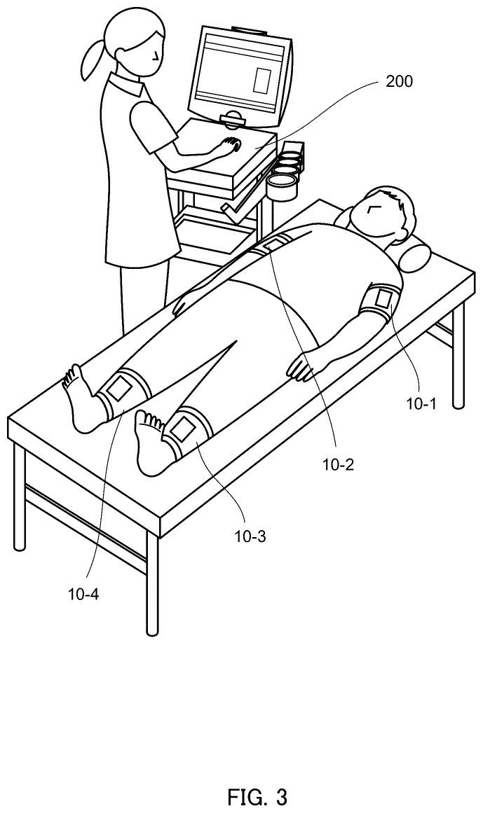

Wireless cuff device 10 is used to perform a blood pressure pulse wave examination. In the present embodiment, four wireless cuff devices 10-1 to 10-4 are used, as illustrated in FIG. 2. As illustrated in FIG. 3, the four wireless cuff devices 10-1 to 10-4 are respectively mounted on the left upper arm, the right upper arm, the left lower limb, and the right lower limb of a subject. In addition to or instead of these mounting sites, for example, when the examination is performed on a toe, a wireless cuff device for a toe may be used. The basic configuration of the wireless cuff device for a toe may be the same as that of wireless cuff device 10 except that the size is different. Conversely, when the blood pressure pulse wave examination is performed by using three or less wireless cuff devices, three or less wireless cuff devices 10 may be used.

The wireless cuff devices 10 (10-1 to 10-4) perform wireless communication with blood pressure pulse wave analyzing apparatus 200 via wireless communication units 100 (100-1 to 100-4). Wireless cuff device 10 inflates and deflates the cuff based on a control signal wirelessly transmitted from blood pressure pulse wave analyzing apparatus 200. In addition, wireless cuff device 10 wirelessly transmits a blood pressure pulse wave measurement result obtained during the examination to blood pressure pulse wave analyzing apparatus 200.

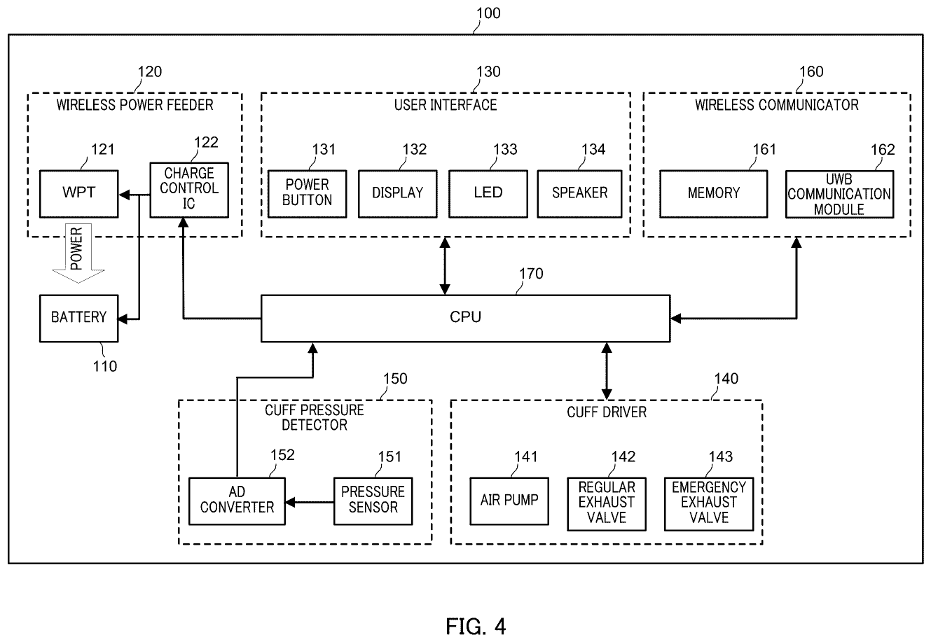

FIG. 4 is a block diagram illustrating a configuration of wireless communication unit 100.

Wireless communication unit 100 includes battery 110, wireless power feeder 120, user interface 130, cuff driver 140, cuff pressure detector 150, wireless communication section (herein a communication section is also referred to as “communicator”) 160, and central processing unit (CPU) 170.

Battery 110 is, for example, a lithium ion secondary battery, and supplies power to an electric circuit in wireless communication unit 100. Wireless power feeder 120 includes wireless power transfer (WPT) 121 composed of a coil or the like, and a charge control integrated circuit (IC) 122 that controls a charging operation of WPT 121 and battery 110. With this configuration, wireless communication unit 100 can perform wireless power feed to battery 110. As battery 110 and wireless power feeder 120, various configurations known in the related art can be adopted.

Wireless communication unit 100 is not necessarily limited to a configuration in which power is fed in a wireless manner, and may have a configuration in which power is fed in a wired manner. However, in hospitals and other facilities where a large number of wireless communication units 100 are used, when wireless communication units 100 are configured to be capable of wireless power supply, it is possible to easily supply power to a large number of wireless communication units 10 detached from cuffs 20, so that the workload of medical workers can be significantly reduced. In addition, in a case of the wired power feed, there is a disadvantage that a contact failure due to aging deterioration or the like may occur, however, in a case of the wireless power feed, there is an advantage that such a contact failure does not occur. Further, since wireless communication unit 100 can be detached from cuff 20 to be charged, wireless communication unit 100 can be charged regardless of whether cuff 20 is in use or not. Therefore, when more wireless communication units 100 than cuffs 20 are prepared, it is not necessary to make a patient wait for charging, and it is not necessary to delay the examination.

User interface 130 includes power button 131, display 132, light-emitting diode (LED) 133, and speaker 134. Power of wireless communication unit 100 is turned on and off by pressing power button 131. Display 132 displays an image indicating an operation state of wireless communication unit 100, an image for the setting, and the like. LED 133 emits light in accordance with the operation state or the alarm state of wireless communication unit 100. An alarm or guidance is output as sound from speaker 134.

Cuff driver 140 includes air pump 141, constant exhaust valve 142, and rapid exhaust valve 143. When wireless communication unit 100 is attached to cuff 20, air pump 141, constant exhaust valve 142, and rapid exhaust valve 143 are connected to the air bag (not illustrated) of cuff 20 via a flow channel (not illustrated).

The air bag of cuff 20 is expanded by air supplied from air pump 141, so that cuff 20 pressurizes the mounting site of the subject. For expanding the air bag, both constant exhaust valve 142 and rapid exhaust valve 143 are controlled to be in a fully closed state. For detecting a pulse wave after avascularization until the blood pressure is determined, constant exhaust valve 142 is opened. In addition, after the blood pressure is determined, rapid exhaust valve 143 is further opened. In this manner, cuff driver 140 controls the cuff pressure during the blood pressure pulse wave examination.

Cuff pressure detector 150 includes pressure sensor 151 and AD converter 152. Pressure sensor 151 is, for example, a pressure-electric conversion sensor composed of a piezo element, and outputs the pressure inside the air bag as an electric signal. AD converter 152 converts the output of pressure sensor 151 into a digital signal and outputs the converted signal to CPU 170. In this manner, the cuff pressure during the blood pressure pulse wave examination is detected by cuff pressure detector 150 and output to CPU 170. The configuration for detecting the cuff pressure is not limited thereto, and various configurations capable of detecting the pressure of the cuff can be adopted.

Wireless communicator 160 includes memory 161 and ultra wide band (UWB) communication module 162. Memory 161 stores identification information, and the identification information is UWB-transmitted by UWB communication module 162. UWB communication module 162 includes an antenna and a transmission/reception circuit that enable a UWB wireless communication method. Memory 161 may be built into CPU 170.

UWB communication module 162 performs UWB wireless communication with blood pressure pulse wave analyzing apparatus 200 to wirelessly receive the control signal from blood pressure pulse wave analyzing apparatus 200 and wirelessly transmit the blood pressure pulse wave measurement result (the blood pressure pulse wave data obtained by cuff pressure detector 150) to blood pressure pulse wave analyzing apparatus 200.

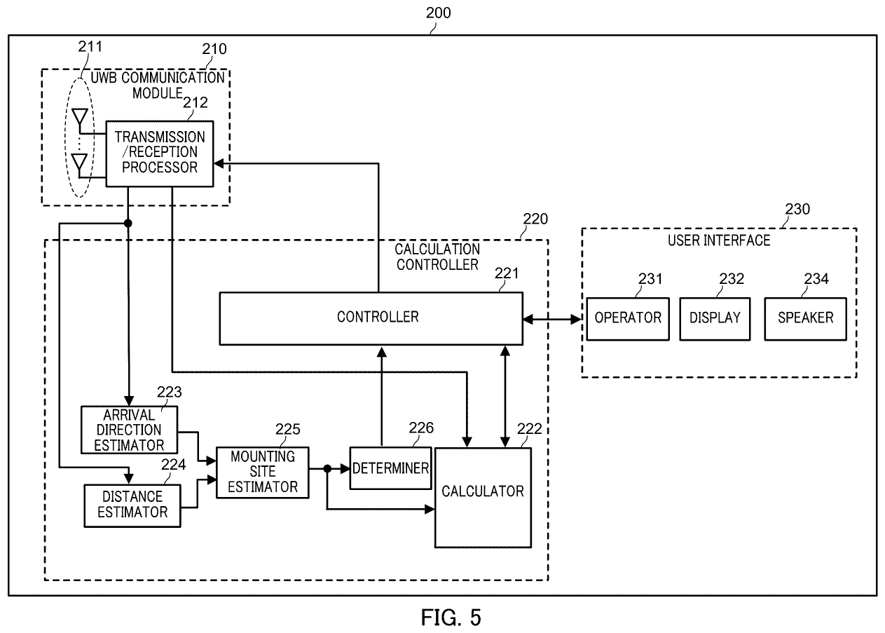

FIG. 5 is a block diagram illustrating a configuration of blood pressure pulse wave analyzing apparatus 200.

Blood pressure pulse wave analyzing apparatus 200 includes UWB communication module 210, calculation controller 220, and user interface 230.

UWB communication module 210 includes antenna 211 composed of a plurality of antenna sections, and transmission/reception processor 212. Transmission/reception processor 212 amplifies and demodulates a reception signal received by antenna 211, and outputs the demodulated signal to calculation controller 220. In addition, transmission/reception processor 212 modulates and amplifies a control signal from calculation controller 220, and outputs the amplified control signal to antenna 211.

Calculation controller 220 includes a central processing unit (CPU), a read only memory (ROM), a random access memory (RAM), and the like as its main components. The CPU reads out a program corresponding to a processing content from the ROM, loads the readout program into the RAM, and executes the operation of each element of calculation controller 220 described below in cooperation with the loaded program. All or a part of calculation controller 220 may be formed by a hardwired circuit, such as an application specific integrated circuit (ASIC) or a field-programmable gate array (FPGA).

Calculation controller 220 includes controller 221 and calculation unit 222.

Controller 221 controls the overall operation of blood pressure pulse wave analyzing apparatus 200. In addition, controller 221 forms a control signal for controlling a plurality of wireless communication units 100-1 to 100-4. The control signal is transmitted to the plurality of wireless communication units 100-1 to 100-4 via UWB communication module 210. As a result, for example, cuff driver 140 of wireless communication unit 100 is controlled by the control signal from controller 221.

Calculation unit 222 receives the blood pressure pulse wave data from the plurality of wireless communication units 100-1 to 100-4 via UWB communication module 210, and calculates a numerical value that serves as an indicator of the state of the blood vessel, such as the PWV or the ABI, based on the blood pressure pulse wave data. Various known processing described in PTL 1 and the like can be applied to the operation executed by calculation unit 222, and therefore, detailed description thereof will be omitted herein. The numerical value serving as the indicator of the state of the blood vessel and obtained by calculation unit 222 is displayed on display 232 of user interface 230.

In addition to such a configuration, calculation controller 220 includes arrival direction estimator 223, distance estimator 224, mounting site estimator 225, and determiner 226.

Arrival direction estimator 223 estimates arrival directions of radio waves from the plurality of wireless cuff devices 10-1 to 10-4. Distance estimator 224 estimates distances to the plurality of wireless cuff devices 10-1 to 10-4. The estimation of the arrival direction and the distance can be achieved by a known position determination function of UWB communication module 210 including the plurality of antennas. For example, the arrival direction can be estimated based on the phase difference of the antennas, and the distance can be estimated based on the difference in reception time.

Mounting site estimator 225 estimates mounting sites of the plurality of wireless cuff devices 10-1 to 10-4 on the subject, based on the arrival directions obtained by arrival direction estimator 223 and the distances obtained by distance estimator 224.

Specifically, as can be seen from FIG. 3, when a positional relationship between the subject and blood pressure pulse wave analyzing apparatus 200 is fixed, it is possible to estimate which of the signals coming from the wireless cuff devices 10-1 to 10-4 corresponds to which of the signals coming from the mounting sites, based on the arrival direction and the distance of the radio wave from each of wireless cuff devices 10-1 to 10-4 with respect to blood pressure pulse wave analyzing apparatus 200.

Mounting site estimator 225 stores the positional relationship between the subject and blood pressure pulse wave analyzing apparatus 200, and mounting site estimator 225 estimates the mounting site (the left upper arm, the right upper arm, the left lower limb, or the right lower limb) for each of wireless cuff devices 10-1 to 10-4 based on the stored positional relationship and the arrival directions and the distances.

In the example of FIG. 3, an example is illustrated in which blood pressure pulse wave analyzing apparatus 200 is disposed on the right side near the head of the subject, however, it is preferable to store a plurality of positional relationships between blood pressure pulse wave analyzing apparatus 200 and the subject in mounting site estimator 225. In this manner, when the user performs a selection operation to select a positional relationship corresponding to an actual positional relationship from among the plurality of stored positional relationships, it is possible to estimate a correct mounting site (the left upper arm, the right upper arm, the left lower limb, or the right lower limb) from the arrival direction and the distance even when the position of the subject with respect to blood pressure pulse wave analyzing apparatus 200 is changed.

In practice, identification (ID) is assigned to memory 161 (FIG. 4) of each of wireless cuff devices 10-1 to 10-4, and mounting site estimator 225 associates the ID with the mounting site. For example, the association is performed as follows: ID1=the left upper arm, ID2=the right upper arm, ID3=the left lower limb, and ID4=the right lower limb.

The result of this association is transmitted to calculation unit 222 and determiner 226. Calculation unit 222 receives the result of the association (for example, ID1=the left upper arm, ID2=the right upper arm, ID3=the left lower limb, and ID4=the right lower limb) from the mounting site estimator 225, and receives the blood pressure pulse wave data (for example, ID1=blood pressure pulse wave data 1, ID2=blood pressure pulse wave data 2, ID3=blood pressure pulse wave data 3, and ID4=blood pressure pulse wave data 4) obtained by wireless cuff devices 10-1 to 10-4 from UWB communication module 210. Calculation unit 222 associates blood pressure pulse wave data 1 with data of the left upper arm, associates blood pressure pulse wave data 2 with data of the right upper arm, associates blood pressure pulse wave data 3 with data of the left lower limb, and associates blood pressure pulse wave data 4 with data of the right lower limb.

Then, calculation unit 222 calculates the numerical value that serves as the indicator of the state of the blood vessel, such as the PWV or the ABI, by using the correspondence relationship. More specifically, calculation unit 222 can obtain, for example, the ABI, the PWV, a brachial-ankle pulse wave velocity (baPWV), a cardio-ankle vascular index (CAVI), and the like based on the blood pressure pulse wave data.

Determiner 226 determines validity of the mounting sites of the plurality of wireless cuff devices 10-1 to 10-4 based on the mounting sites estimated by mounting site estimator 225. Here, as described above, wireless cuff devices 10-1 to 10-4 are colored according to the respective mounting sites, and the user attaches wireless cuff devices 10-1 to 10-4 on the respective mounting sites (the left upper arm, the right upper arm, the left lower limb, and the right lower limb) corresponding to the colors of the cuffs.

When wireless cuff devices 10-1 to 10-4 are respectively mounted on the correct mounting sites by the user, the following should be satisfied: ID1=the left upper arm, ID2=the right upper arm, ID3=the left lower limb, and ID4=the right lower limb. However, when the estimation result, for example, ID1=the left lower limb, ID2=the right upper arm, ID3=the left upper arm, and ID4=the right lower limb is obtained by mounting site estimator 225, there is a high possibility that wireless cuff device 10-1 for the left upper arm and wireless cuff device 10-3 for the left lower limb are incorrectly mounted.

Determiner 226 determines the validity of the mounting sites of the plurality of wireless cuff devices 10-1 to 10-4 by comparing a predetermined relationship of the mounting sites with a relationship of the mounting sites obtained by mounting site estimator 225. Determiner 226 outputs the determination result to controller 221. When the determination result indicating that wireless cuff devices 10-1 to 10-4 are mounted on the incorrect parts is input, controller 221 outputs, for example, an alarm indicating that the mounting sites are incorrect from display 232 and/or speaker 234 of user interface 230.

Here, in the configuration of the present embodiment, even when the mounting sites of wireless cuff devices 10-1 to 10-4 are incorrect, calculation unit 222 can calculate the numerical value serving as the indicator of the state of the blood vessel, such as the PWV or the ABI, with the correct correspondence relationship due to the association by mounting site estimator 225. However, since there is a case where the size or the measurement sensitivity is different between the wireless cuff device for an upper arm and the wireless cuff device for a lower limb, it is preferable that each wireless cuff device is mounted on a predetermined site. In the present embodiment, by providing determiner 226, incorrect cuff attachment by the user can be corrected, thereby preventing a decrease in accuracy of the blood pressure pulse wave examination due to incorrect cuff attachment.

As described above, according to the present embodiment, the blood pressure pulse wave analyzing system includes the following: a plurality of wireless cuff devices 10-1 to 10-4 to be mounted on predetermined sites of the subject; and blood pressure pulse wave analyzing apparatus 200 that performs wireless communication with the plurality of wireless cuff devices 10-1 to 10-4, controls the plurality of wireless cuff devices 10-1 to 10-4, and collects the blood pressure pulse wave data from the plurality of wireless cuff devices 10-1 to 10-4, thereby examining the blood vessel state of the subject. Each of the plurality of wireless cuff devices 10-1 to 10-4 includes the following: cuff 20 including an air bag and to be wound around the corresponding predetermined site of the subject; and wireless communication unit 100 attached to cuff 20. Blood pressure pulse wave analyzing apparatus 200 includes the following: arrival direction estimator 223 that estimates the arrival directions of the radio waves from the plurality of wireless cuff devices 10-1 to 10-4; distance estimator 224 that estimates the distances to the plurality of wireless cuff devices 10-1 to 10-4; and mounting site estimator 225 that estimates the mounting sites of the plurality of wireless cuff devices 10-1 to 10-4 on the subject based on the estimated arrival directions and distances.

As a result, it is possible to achieve the blood pressure pulse wave analyzing system and blood pressure pulse wave analyzing apparatus 200 that can achieve the hoseless configuration.

### <1-3> Another Form in Embodiment 1

### <1-3-1> Another Form 1

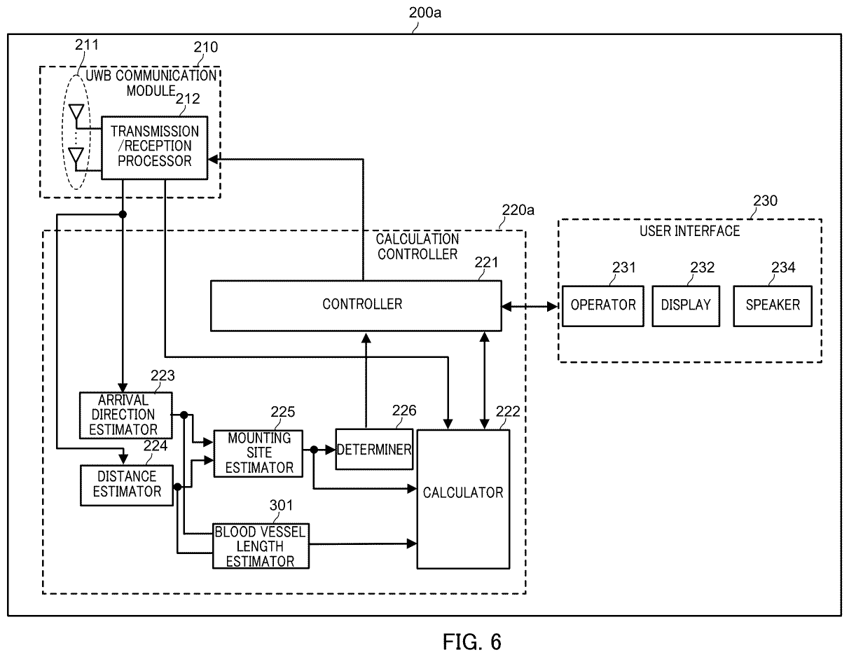

FIG. 6 is a block diagram illustrating a configuration of blood pressure pulse wave analyzing apparatus 200a according to the other form 1 in Embodiment 1, and in FIG. 6, parts corresponding to those in FIG. 5 are represented by the same reference numerals.

Blood pressure pulse wave analyzing apparatus 200a includes, in addition to the configuration of blood pressure pulse wave analyzing apparatus 200 according to the above-described embodiment, the following: blood vessel length estimator 301 that estimates a blood vessel length of a subject based on an arrival direction estimated by arrival direction estimator 223 and a distance estimated by distance estimator 224; and calculation unit 222 that calculates an indicator (for example, PWV, baPWV, CAVI, or the like) indicating the blood vessel state of the subject based on the estimated blood vessel length and the blood pressure pulse wave data from the plurality of wireless cuff devices 10-1 to 10-4.

Here, in calculation unit 222, in order to calculate the PWV, the baPWV, the CAVI, or the like as the indicator indicating the blood vessel state of a subject, information on the blood vessel length of the subject is necessary. Specifically, the blood vessel length from the heart to each of wireless cuff devices 10-1 to 10-4 is necessary. Since it is difficult to actually measure this blood vessel length, in the related art, a medical worker inputs the height or the like of the subject to blood pressure pulse wave analyzing apparatus 200, and calculation unit 222 virtually obtains the blood vessel length from the value of the height.

On the other hand, in blood pressure pulse wave analyzing apparatus 200a according to the present embodiment, the blood vessel length of a subject is estimated by blood vessel length estimator 301, so that it is not necessary to input the height of the subject. As a result, it is possible to achieve a blood pressure pulse wave analyzing system and a blood pressure pulse wave analyzing apparatus capable of improving convenience when the hoseless configuration is achieved.

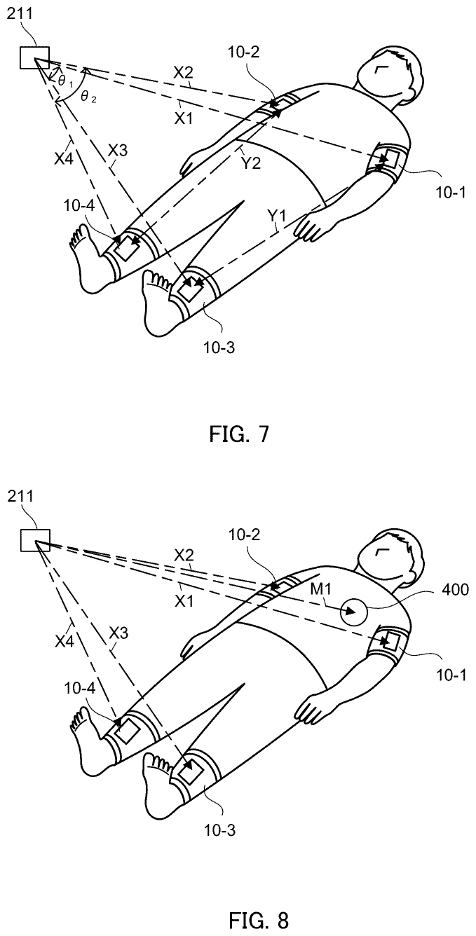

FIG. 7 is a diagram for describing estimation of a blood vessel length by blood vessel length estimator 301.

Distance estimator 224 acquires distances X1, X2, X3, and X4 to wireless cuff devices 10-1, 10-2, 10-3, and 10-4, respectively. In addition, angles θ1 and θ2 in the drawing can be acquired based on the directions of wireless cuff devices 10-1, 10-2, 10-3, and 10-4 estimated by arrival direction estimator 223.

Blood vessel length estimator 301 obtains distances Y1 and Y2 in the drawing based on distances X1, X2, X3, and X4 and angles θ1 and θ2. Distances Y1 and Y2 can be obtained using the law of cosines. Distances Y1 and Y2 are lengths corresponding to the height or the like of the subject, and therefore, blood vessel length estimator 301 can obtain the blood vessel length from the heart of the subject to each of wireless cuff devices 10-1 to 10-4 as an estimation value by substituting distance Y1 or Y2 into a predetermined model expression.

One aspect of the blood pressure pulse wave analyzing system according to the present embodiment includes the following: the plurality of wireless cuff devices 10-1 to 10-4 to be mounted on the predetermined sites of the subject; and blood pressure pulse wave analyzing apparatus 200a that performs wireless communication with the plurality of wireless cuff devices 10-1 to 10-4, controls the plurality of wireless cuff devices 10-1 to 10-4, and collects the blood pressure pulse wave data from the plurality of wireless cuff devices 10-1 to 10-4, thereby examining the blood vessel state of the subject. Each of the plurality of wireless cuff devices 10-1 to 10-4 includes the following: cuff 20 including an air bag and to be wound around the corresponding predetermined site of the subject, and wireless communication unit 100 attached to cuff 20. Blood pressure pulse wave analyzing apparatus 200a includes the following: arrival direction estimator 223 that estimates the arrival directions of the radio waves from the plurality of wireless cuff devices 10-1 to 10-4; distance estimator 224 that estimates the distances to the plurality of wireless cuff devices 10-1 to 10-4; blood vessel length estimator 301 that estimates a blood vessel length of the subject based on the estimated arrival directions and distances; and calculation unit 222 that calculates an indicator indicating the blood vessel state of the subject based on the estimated blood vessel length and the blood pressure pulse wave data from the plurality of wireless cuff devices 10-1 to 10-4.

### <1-3-2> Another Form 2

In the other form 2, it is proposed to implement a wireless heart sound sensor. That is, a heart sound sensor that can be wirelessly connected to blood pressure pulse wave analyzing apparatus 200a is used. Specifically, the heart sound sensor includes a wireless transmitter that can wirelessly transmit a detected heart sound signal to blood pressure pulse wave analyzing apparatus 200a. Blood pressure pulse wave analyzing apparatus 200a calculates an indicator indicating the blood vessel state of the subject, such as the PWV, the baPWV, or the CAVI, based on the heart sound signal from the heart sound sensor in addition to the blood pressure pulse wave data from the plurality of wireless cuff devices 10-1 to 10-4.

A method of calculating the indicator indicating the blood vessel state of the subject, such as the PWV, the baPWV, or the CAVI, by using the blood pressure pulse wave data and heart sounds has been widely performed in the related art, and therefore, the description thereof will be omitted herein.

A feature of the present embodiment is that blood pressure pulse wave analyzing apparatus 200a estimates the blood vessel length from the heart to each of wireless cuff devices 10-1 to 10-4 by using the wireless heart sound sensor. In this manner, the accuracy of estimating the blood vessel length is improved as compared with Embodiment 1, and the indicator (PWV, baPWV, CAVI, or the like) indicating the blood vessel state of the subject can be obtained more accurately.

FIG. 8 is a diagram for describing estimation of a blood vessel length by blood vessel length estimator 301.

Distance estimator 224 acquires distance M1 to wireless heart sound sensor 400 in addition to distances X1, X2, X3, and X4 to wireless cuff devices 10-1, 10-2, 10-3, and 10-4, respectively. In addition, arrival direction estimator 223 acquires the direction of wireless heart sound sensor 400 in addition to the directions of wireless cuff devices 10-1, 10-2, 10-3, and 10-4.

Blood vessel length estimator 301 obtains the distances from the wireless heart sound sensor 400 to respective wireless cuff devices 10-1, 10-2, 10-3, and 10-4 based on distances X1, X2, X3, X4, and M1 and the directions of wireless cuff devices 10-1, 10-2, 10-3, and 10-4, and wireless heart sound sensor 400. The distances from wireless heart sound sensor 400 to respective wireless cuff devices 10-1, 10-2, 10-3, and 10-4 can be obtained by using the law of cosines.

Blood vessel length estimator 301 can obtain the blood vessel length from the heart of the subject to each of wireless cuff devices 10-1 to 10-4 as the estimation value by substituting the obtained distance from wireless heart sound sensor 400 to each of wireless cuff devices 10-1, 10-2, 10-3, and 10-4 into a predetermined model expression. For example, a blood vessel from wireless heart sound sensor 400 to each of wireless cuff devices 10-1, 10-2, 10-3, and 10-4, that is, a blood vessel from the heart to a blood pressure pulse wave measurement point extends from the heart to the blood pressure pulse wave measurement point in an arched shape. Therefore, blood vessel length estimator 301 obtains the estimation value of the blood vessel length by performing correction of converting a straight-line distance into a curved distance of an arch shape. In the present embodiment, the blood vessel length from the heart to each of wireless cuff devices 10-1 to 10-4 is estimated with reference to the position of wireless heart sound sensor 400 corresponding to the position of the heart, so that the accuracy of estimating the blood vessel length is improved as compared with Embodiment 1, and the indicator (PWV, baPWV, CAVI, or the like) indicating the blood vessel state of the subject can be obtained more accurately.

One aspect of a blood pressure pulse wave analyzing system according to the other form 2 includes the following: the plurality of wireless cuff devices 10-1 to 10-4 to be mounted on the predetermined sites of a subject; blood pressure pulse wave analyzing apparatus 200a that performs wireless communication with the plurality of wireless cuff devices 10-1 to 10-4, controls the plurality of wireless cuff devices 10-1 to 10-4, and collects the blood pressure pulse wave data from the plurality of wireless cuff devices 10-1 to 10-4, thereby examining the blood vessel state of the subject; and wireless heart sound sensor 400.

Blood pressure pulse wave analyzing apparatus 200a includes the following: arrival direction estimator 223 that estimates the arrival directions of the radio waves from the plurality of wireless cuff devices 10-1 to 10-4 and the arrival direction of the radio wave from wireless heart sound sensor 400; distance estimator 224 that estimates the distances to the plurality of wireless cuff devices 10-1 to 10-4 and to wireless heart sound sensor 400; blood vessel length estimator 301 that estimates blood vessel lengths based on the distances from wireless heart sound sensor 400 to the plurality of wireless cuff devices 10-1 to 10-4; and calculation unit 222 that calculates an indicator indicating the blood vessel state of the subject based on the estimated blood vessel lengths and the blood pressure pulse wave data from the plurality of wireless cuff devices 10-1 to 10-4.

In the other form 2, the example has been described in which wireless heart sound sensor 400 is used, however, another wireless sensor may be used instead of wireless heart sound sensor 400. In short, it is sufficient to provide a wireless sensor including a sensor that can detect the behavior of the heart, which is a source of the pulse wave, from the heart, and a wireless section that wirelessly transmits the acquired heart behavior information to blood pressure pulse wave analyzing apparatus 200a. Here, the heart behavior information is, specifically, information for obtaining the ejection timing of the blood from the heart on the blood pressure pulse wave analyzing apparatus 200a side. The sensor section for detecting the behavior of the heart may be, for example, a vibration sensor that detects chest vibrations in response to the beating of the heart. In addition, the sensor section can be a sensor that measures an electrocardiogram.

In addition, the other form 2 describes the case in which the total of four wireless cuff devices 10-1 to 10-4 are mounted on the upper limbs and the lower limbs, however, the number and the positions of the mounted wireless cuff devices are not limited thereto. When the pulse wave propagation velocity is measured by using the wireless sensor (for example, wireless heart sound sensor 400) attached to the body surface corresponding to the heart, the number of wireless cuff devices may be, for example, one. As described in section <1-3-2>, when the pulse wave propagation velocity is measured without using the wireless sensor (for example, wireless heart sound sensor 400), the plurality of wireless cuff devices are required. The plurality of wireless cuff devices are mounted on, for example, the upper arm and the other upper limb and/or a lower limb. Alternatively, for example, the wireless cuff devices may be mounted on two different positions on the lower limbs as in a case of a segmental pulse wave examination. These two wireless cuff devices may be mounted at positions corresponding to both ends of the artery that do not straddle a branching point. The reason for this is common in the field of measuring the pulse wave propagation velocity in the related art, and thus the description thereof will be omitted herein.

When the estimation of a blood vessel length is performed as in other forms 1 and 2, it is preferable that antenna 211 is installed on a lower side of a bed on which the subject lies. For example, antenna 211 is installed to be integrated with the bed or is installed to be integrated with the sheet. In this manner, for example, angles θ1 and θ2 in FIG. 7 are not excessively small, so that it is possible to prevent a decrease in the measurement accuracy of distances Y1 and Y2.

### <1-3-3> Another Form 3

The other forms 1 and 2 describe the case in which blood pressure pulse wave analyzing apparatus 200a estimates the distances to and the directions of wireless cuff devices 10-1 to 10-4 and wireless heart sound sensor 400, and estimates a blood vessel length based on the estimated distances and directions, however, wireless cuff devices 10-1 to 10-4 and wireless heart sound sensor 400 may perform distance measurement by wireless communication, and a result of the distance measurement may be transmitted to blood pressure pulse wave analyzing apparatus 200a.

For example, in FIG. 7, wireless cuff device 10-1 and wireless cuff device 10-3 perform the distance measurement by wireless communication to obtain distance Y1, wireless cuff device 10-2 and wireless cuff device 10-4 perform the distance measurement by wireless communication to obtain distance Y2, and wireless cuff devices 10-1 to 10-4 transmit the information on distances Y1 and Y2 to blood pressure pulse wave analyzing apparatus 200a. Blood pressure pulse wave analyzing apparatus 200a receives the information on distances Y1 and Y2 and estimates the blood vessel lengths between wireless cuff devices 10-1 to 10-4 based on the information on distances Y1 and Y2.

In addition, for example, in FIG. 8, wireless heart sound sensor 400 and each of wireless cuff devices 10-1 to 10-4 perform the distance measurement by wireless communication to obtain the distances from wireless heart sound sensor 400 to respective wireless cuff devices 10-1 to 10-4, and wireless heart sound sensor 400 or wireless cuff devices 10-1 to 10-4 transmit the information on the distances to blood pressure pulse wave analyzing apparatus 200a. Blood pressure pulse wave analyzing apparatus 200a receives the information on the distances and estimates the blood vessel lengths from wireless heart sound sensor 400 to respective wireless cuff devices 10-1 to 10-4 based on the information on the distances.

In the present embodiment, arrival direction estimator 223 and distance estimator 224 of blood pressure pulse wave analyzing apparatus 200a illustrated in FIG. 6 can be omitted. In addition, antenna 211 and the like can be configured not to have the position determination function.

### <1-3-4> Another Form 4

In addition to the above-described embodiments, controller 221 may pair with a wireless cuff device (from the plurality of wireless cuff devices) located within a predetermined distance range, based on the information on the distance to each wireless cuff device estimated by distance estimator 224, and may collect the blood pressure pulse wave data from the wireless cuff device located within the predetermined distance range. In this manner, even when more than one blood pressure pulse wave analyzing system as illustrated in FIG. 3 is present nearby (for example, within 5 m from each other or in the same examination room), it is possible to prevent synchronization (interference) with a wireless cuff device of another blood pressure pulse wave analyzing system located nearby.

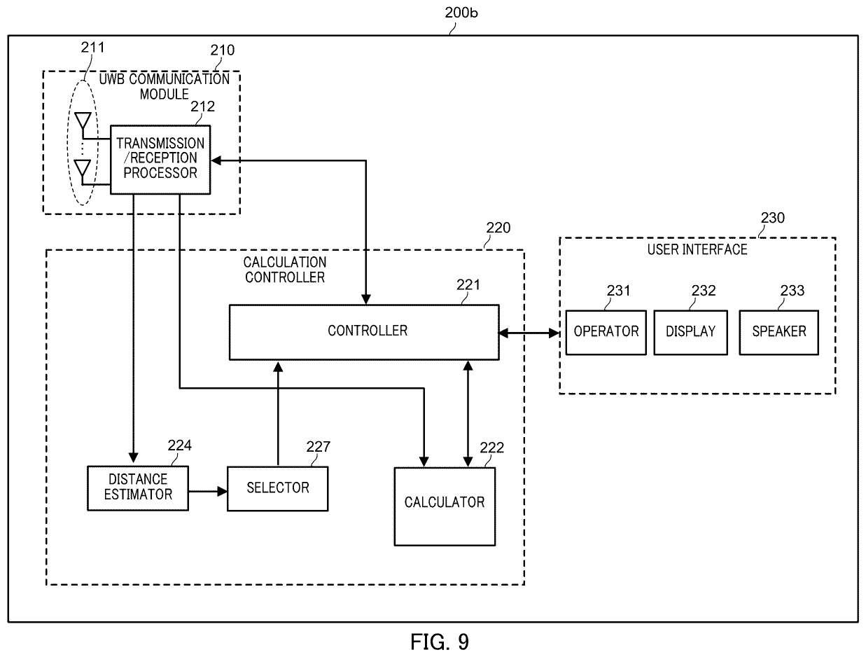

Based on this concept, a configuration as illustrated in FIG. 9 may be adopted. As compared with blood pressure pulse wave analyzing apparatus 200 of FIG. 5, arrival direction estimator 223, mounting site estimator 225, and determiner 226 are omitted from blood pressure pulse wave analyzing apparatus 200b. Blood pressure pulse wave analyzing apparatus 200b includes distance estimator 224 that estimates the distances to the plurality of wireless cuff devices, selector 227 that selects a wireless cuff device located within the predetermined distance range based on the estimated distances, and calculation unit 222 that calculates an indicator indicating the blood vessel state of the subject based on the blood pressure pulse wave data from the wireless cuff device selected by selector 227.

Controller 221 controls transmission/reception processor 212 so as to pair with the wireless cuff device selected by selector 227. In other words, controller 221 excludes, from the pairing target, a wireless cuff device other than the wireless cuff device selected by selector 227, even when a wireless signal from the excluded wireless cuff device can be received. For example, even when the reception power from a certain wireless cuff device is large, controller 221 excludes the wireless cuff device from the pairing target when the distance to the wireless cuff device is larger than the predetermined distance.

In the configuration of FIG. 9, as compared with the configuration of FIG. 5, the mounting site cannot be specified, and thus it is a precondition that the wireless cuff should be mounted on the correct mounting site by the user. However, there is an advantage in that, even when another blood pressure pulse wave analyzing system is present nearby (for example, within 5 m from each other or in the same examination room), it is possible to prevent synchronization (interference) with a wireless cuff device of the other blood pressure pulse wave analyzing system.

In addition, even when wireless cuff devices 10-1 to 10-4 are collectively placed at one place near blood pressure pulse wave analyzing apparatus 200b before being mounted on the subject, all wireless cuff devices 10-1 to 10-4 can be collectively paired based on the distance information.

In addition, blood pressure pulse wave analyzing apparatus 200b according to the other form 4 pairs only with a wireless cuff device within a predetermined distance and communicates with the wireless cuff device, and therefore, it is possible to prevent “falsification of the blood pressure pulse wave data” through impersonation and unauthorized access to blood pressure pulse wave analyzing apparatus 200b, thereby improving cyber security.

In the blood pressure pulse wave analyzing system and the blood pressure pulse wave examination according to the other form 4, the number of wireless cuff devices 10 is not limited to four, and the blood pressure pulse wave analyzing system and the blood pressure pulse wave examination can be widely applied when one or more wireless cuff devices 10 are provided, as in the other form 2.

Embodiment 1 and the other forms 1 to 4 can also be implemented in combination with each other. In addition, Embodiment 1 describes the case in which blood pressure pulse wave analyzing apparatus 200 examines the state of a blood vessel by using blood pressure pulse waves detected by wireless cuff devices 10-1 to 10-4, however, blood pressure pulse wave analyzing apparatus 200 may examine the state of the blood vessel by using the heart sound in addition to the blood pressure pulse waves. In this case, the blood pressure pulse wave analyzing system may be configured to include a heart sound sensor in addition to the configuration of FIG. 3. In this case, blood pressure pulse wave analyzing apparatus 200 and the heart sound sensor are connected to each other in a wireless or wired manner. The examination of the blood vessel using the blood pressure pulse wave and the heart sound is a known technique described in, for example, PTL 1, and thus the description thereof will be omitted herein.

Embodiment 1 and the other forms 1 to 3 describe the case in which UWB is used as the wireless method, however, the wireless method to be applied is not limited to UWB. For example, Bluetooth (registered trademark) may be used. However, the use of UWB has the advantage that the estimation accuracy of the arrival direction and distance can be increased, making it possible to more accurately determine an indicator (PWV, baPWV, CAVI, or the like) indicating the blood vessel state of the subject.

In Embodiment 1 and the other forms 1 to 3, the term “blood pressure pulse wave data” may be replaced with “pulse wave data”. Specifically, the configuration of the above-described embodiments can also be applied to a pulse wave analyzing apparatus that obtains an indicator of the arterial stiffness based on the pulse wave data without using the blood pressure data. The same applies to all embodiments described below.

### <1-4> Conclusion of Embodiment 1 and Other Forms 1 to 4

(1) One aspect of a blood pressure pulse wave analyzing system according to the present disclosure includes the following:
a plurality of wireless cuff devices each to be mounted on a predetermined site of a subject; and
a blood pressure pulse wave analyzing apparatus that performs wireless communication with the plurality of wireless cuff devices, controls the plurality of wireless cuff devices, and collects pulse wave data from the plurality of wireless cuff devices to examine a blood vessel state of the subject, in which
each of the plurality of wireless cuff devices includes
   a cuff including an air bag and to be wound around the predetermined site of the subject, and
   a wireless communication unit attached to the cuff, and
the blood pressure pulse wave analyzing apparatus includes
   an arrival direction estimator that estimates an arrival direction of a radio wave from each of the plurality of wireless cuff devices,
   a distance estimator that estimates a distance to each of the plurality of wireless cuff devices, and
   a mounting site estimator that estimates a mounting site of each of the plurality of wireless cuff devices on the subject based on the estimated arrival direction and the estimated distance.
(2) One aspect of the blood pressure pulse wave analyzing system according to the present disclosure is as follows in the aspect (1):
the blood pressure pulse wave analyzing apparatus further includes
   a determiner that determines validity of the mounting site of each of the plurality of wireless cuff devices based on the estimated mounting site, and
   an output that outputs a determination result of the determiner.
(3) One aspect of the blood pressure pulse wave analyzing system according to the present disclosure is as follows in the aspect (1) or (2):
   the blood pressure pulse wave analyzing apparatus and the plurality of wireless cuff devices each include a UWB communicator; and
the arrival direction estimator and the distance estimator of the blood pressure pulse wave analyzing apparatus estimate the arrival direction and the distance based on radio waves in a UWB communication method.
(4) One aspect of a blood pressure pulse wave analyzing apparatus according to the present disclosure includes the following:
an arrival direction estimator that estimates an arrival direction of a radio wave from each of the plurality of wireless cuff devices,
a distance estimator that estimates a distance to each of the plurality of wireless cuff devices, and
a mounting site estimator that estimates mounting sites of the plurality of wireless cuff devices on the subject based on the estimated arrival direction and distances.
(5) One aspect of the blood pressure pulse wave analyzing apparatus according to the present disclosure further includes the following in the aspect (4):
a determiner that determines validity of the mounting site of each of the plurality of wireless cuff devices based on the estimated mounting site; and
an output that outputs a determination result of the determiner.
(6) One aspect of the blood pressure pulse wave analyzing apparatus according to the present disclosure further includes the following in the aspect (4):
a UWB communicator, and
the arrival direction estimator and the distance estimator estimate the arrival direction and the distance based on radio waves in a UWB communication method.
(7) One aspect of a blood pressure pulse wave analyzing system according to the present disclosure includes the following:
a wireless cuff device to be mounted on a predetermined site of a subject; and
a blood pressure pulse wave analyzing apparatus that performs wireless communication with the wireless cuff device, controls the wireless cuff device, and collects pulse wave data from the wireless cuff device to examine a blood vessel state of the subject, in which
the wireless cuff device includes
   a cuff that includes an air bag and is to be wound around the predetermined site of the subject, and
   a wireless communication unit attached to the cuff, and
the blood pressure pulse wave analyzing apparatus includes
   a distance estimator that estimates a distance to the wireless cuff device,
   a selector that selects the wireless cuff device located within a predetermined distance range based on the estimated distance, and
   a calculation unit that calculates an indicator indicating the blood vessel state of the subject based on the pulse wave data from the wireless cuff device selected by the selector.
(8) One aspect of the blood pressure pulse wave analyzing system according to the present disclosure is as follows in the aspect (7), the blood pressure pulse wave analyzing apparatus pairs with the wireless cuff device selected by the selector.
(9) One aspect of the blood pressure pulse wave analyzing apparatus according to the present disclosure is as follows in the aspect (7):
the blood pressure pulse wave analyzing apparatus excludes, from the pairing target, a wireless cuff device other than the wireless cuff device selected by the selector even when a wireless signal from the excluded wireless cuff device can be received.
(10) One aspect of a blood pressure pulse wave analyzing system according to the present disclosure includes the following:
a distance estimator that estimates a distance to each of a plurality of wireless cuff devices,
a selector that selects a wireless cuff device located within a predetermined distance range based on the estimated distance, the wireless cuff device being among the plurality of wireless cuff devices, and
a calculation unit that calculates an indicator indicating a blood vessel state of a subject based on pulse wave data from the wireless cuff device selected by the selector.
(11) One aspect of the blood pressure pulse wave analyzing apparatus according to the present disclosure further includes the following in the aspect (10):
a controller that controls the wireless communicator to form a pair with the wireless cuff device selected by the selector.
(12) One aspect of the blood pressure pulse wave analyzing apparatus according to the present disclosure further includes the following in the aspect (10):
a controller that excludes, from a pairing target, a wireless cuff device other than the wireless cuff device selected by the selector even when a wireless signal from the excluded wireless cuff device can be received.
(13) One aspect of a blood pressure pulse wave analyzing system according to the present disclosure includes the following:
a wireless cuff device to be mounted on a predetermined site of a subject; and
a blood pressure pulse wave analyzing apparatus that performs wireless communication with the wireless cuff device, controls the wireless cuff device, and collects pulse wave data from the wireless cuff device to examine a blood vessel state of the subject, in which,
the position of the wireless cuff device is wirelessly determined, and
the blood pressure pulse wave analyzing apparatus estimates a blood vessel length of the subject by using a result of determination of the position, and calculates an indicator indicating the blood vessel state of the subject based on the estimated blood vessel length and the pulse wave data from the wireless cuff device.
(14) One aspect of the blood pressure pulse wave analyzing system according to the present disclosure includes the following in the aspect (13):
a plurality of the wireless cuff device, in which
the blood pressure pulse wave analyzing apparatus includes
   an arrival direction estimator that estimates an arrival direction of a radio wave from each of the plurality of wireless cuff devices,
   a distance estimator that estimates a distance to each of the plurality of wireless cuff devices,
   a blood vessel length estimator that estimates a blood vessel length between the plurality of wireless cuff devices of the subject based on the estimated arrival direction and the estimated distance regarding the plurality of wireless cuff devices, and
   a calculation unit that calculates an indicator indicating the blood vessel state of the subject based on the estimated blood vessel length and the pulse wave data from the plurality of wireless cuff devices.
(15) One aspect of the blood pressure pulse wave analyzing system according to the present disclosure further includes the following in the aspect (13):
a wireless sensor that is mounted on the subject at a position corresponding to a heart of the subject, detects a behavior of the heart, and wirelessly transmits heart behavior information, the behavior being a source of a pulse wave, in which
the blood pressure pulse wave analyzing apparatus includes
   an arrival direction estimator that estimates arrival directions of radio waves from the wireless cuff device and the wireless sensor,
a distance estimator that estimates a distance between the wireless cuff device and the wireless sensor,
a blood vessel length estimator that estimates a blood vessel length of the subject between the wireless cuff device and the wireless sensor based on the estimated arrival directions and the estimated distance regarding the wireless cuff device and the wireless sensor, and
a calculation unit that calculates an indicator indicating the blood vessel state of the subject based on the estimated blood vessel length, the pulse wave data, and the heart behavior information.
(16) One aspect of the blood pressure pulse wave analyzing system according to the present disclosure includes the following in the aspect (13):
a plurality of the wireless cuff devices, in which
the plurality of wireless cuff devices obtain a distance between the plurality of wireless cuff devices wirelessly, and wirelessly transmit information on the distance to the blood pressure pulse wave analyzing apparatus.
(17) One aspect of the blood pressure pulse wave analyzing system according to the present disclosure further includes the following in the aspect (13):
a wireless sensor that is mounted on the subject at a position corresponding to a heart of the subject, detects a behavior of the heart, and wirelessly transmits heart behavior information, the behavior being a source of a pulse wave, in which
the wireless sensor and the wireless cuff device obtain a distance between the wireless sensor and the wireless cuff device wirelessly, and wirelessly transmit information on the distance to the blood pressure pulse wave analyzing apparatus.
(18) One aspect of a blood pressure pulse wave analyzing apparatus according to the present disclosure includes the following:
an arrival direction estimator that estimates an arrival direction of a radio wave from each of a plurality of wireless cuff devices mounted on a predetermined site of a subject,
a distance estimator that estimates a distance to each of the plurality of wireless cuff devices,
a blood vessel length estimator that estimates a blood vessel length between the plurality of wireless cuff devices on the subject based on the estimated arrival direction and distance regarding the plurality of wireless cuff devices, and
a calculation unit that calculates an indicator indicating a blood vessel state of the subject based on the estimated blood vessel length and pulse wave data from the plurality of wireless cuff devices.
(19) One aspect of a blood pressure pulse wave analyzing apparatus according to the present disclosure includes the following:
an arrival direction estimator that estimates an arrival direction of a radio wave from a wireless cuff device mounted on a predetermined site of a subject and an arrival direction of a radio wave from a wireless sensor that is mounted on the subject at a position corresponding a heart of the subject, detects a behavior of the heart (which is a source of a pulse wave), and wirelessly transmits heart behavior information;
a distance estimator that estimates a distance between the wireless cuff device and the wireless sensor;
a blood vessel length estimator that estimates a blood vessel length of the subject between the wireless cuff device and the wireless sensor based on the estimated arrival directions and the estimated distance regarding the wireless cuff device and the wireless sensor; and
a calculation unit that calculates an indicator indicating a blood vessel state of the subject based on the estimated blood vessel length, pulse wave data from the wireless cuff device, and the heart behavior information from the wireless sensor.

### <2> Embodiment 2

### <2-1> Configuration of Embodiment 2

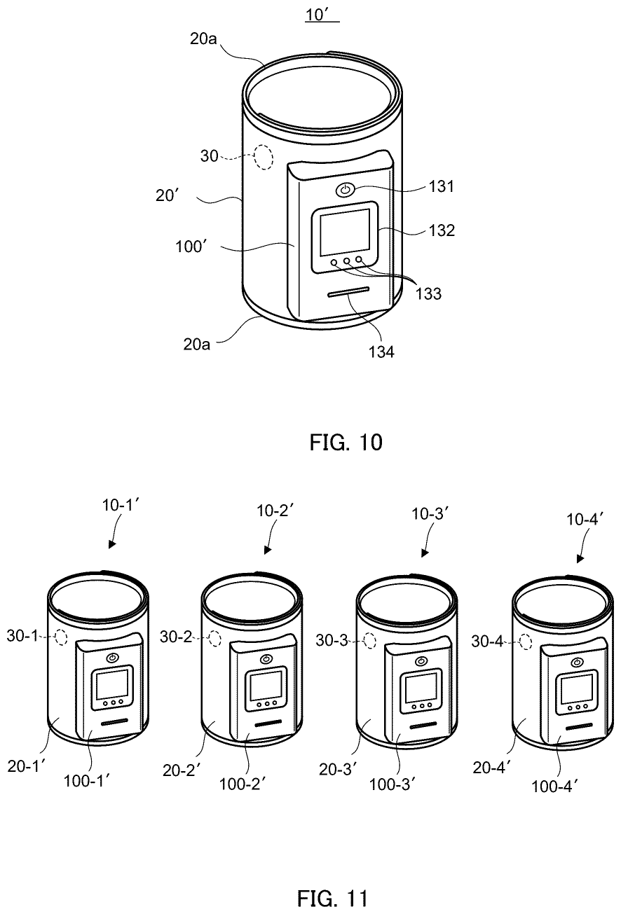

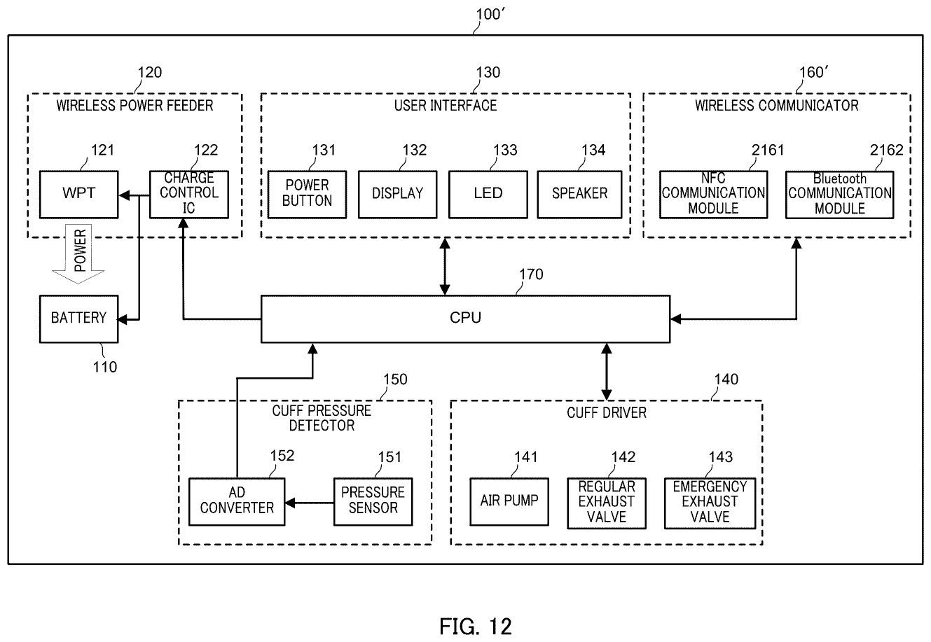

FIG. 10 is a perspective view illustrating a configuration of wireless cuff device 10′ of the present embodiment, and in FIG. 10, parts corresponding to those in FIG. 1 are represented by the same reference numerals. Wireless cuff device 10′ of the present embodiment is configured such that wireless tag 30 is provided in cuff 20′ as compared with wireless cuff device 10 of Embodiment 1. Wireless tag 30 is provided at a distance at which wireless tag 30 can communicate with wireless communication unit 100′. In the present embodiment, wireless tag 30 is a near field communication (NFC) tag. The communicable distance of an NFC tag is generally less than 10 cm, and therefore, wireless tag 30 is provided at a distance of less than 10 cm from NFC communication module 2161 (FIG. 12) of wireless communication unit 100′. The NFC tag has a short range and strong directivity, and therefore, interference with wireless devices such as another NFC tag in the periphery can be minimized.

Wireless tag 30 includes an NFC communication module that achieves NFC communication with wireless communication unit 100′ and a memory. The memory stores information on a mounting site (for example, the left upper arm, the right upper arm, the left lower limb, or the right lower limb) where cuff 20′ (to which wireless tag 30 is attached) is mounted. In addition, the memory stores information indicating the size of cuff 20′ to which wireless tag 30 is attached (for example, information indicating which size the cuff is, S, M, or L) and/or information indicating the type of cuff 20′ (for an adult, a child, or an infant).

Wireless cuff device 10′ is used to perform the blood pressure pulse wave examination. In the present embodiment, as illustrated in FIG. 11, four wireless cuff devices 10-1′ to 10-4′ are used. As illustrated in FIG. 3, four wireless cuff devices 10-1′ to 10-4′ are respectively mounted on the left upper arm, the right upper arm, the left lower limb, and the right lower limb of the subject (suffice it to say that 10-1 to 10-4 in FIG. 3 are replaced with 10-1′ to 10-4′, respectively). In addition to or instead of these mounting sites, for example, when the examination is performed on a toe, a wireless cuff device for a toe may be used. The basic configuration of the wireless cuff device for a toe may be the same as that of wireless cuff device 10′ except that the size is different. Conversely, when the blood pressure pulse wave examination is performed by using three or less wireless cuff devices, three or less wireless cuff devices 10′ may be used.

Wireless cuff devices 10′ (10-1′ to 10-4′) perform wireless communication with blood pressure pulse wave analyzing apparatus 200 (FIG. 5) via wireless communication units 100′ (100-1′ to 100-4′). Wireless cuff device 10′ inflates and deflates the cuff based on a control signal wirelessly transmitted from blood pressure pulse wave analyzing apparatus 200. In addition, wireless cuff device 10′ wirelessly transmits a blood pressure pulse wave measurement result obtained during the examination to blood pressure pulse wave analyzing apparatus 200.

FIG. 12 is a block diagram illustrating a configuration of wireless communication unit 100′ of Embodiment 2, and in FIG. 12, parts corresponding to those in FIG. 4 are represented by the same reference numerals. Wireless communication unit 100′ of the present embodiment has a different configuration of wireless communicator 160′ as compared with wireless communication unit 100 of Embodiment 1.

Wireless communicator 160′ of the present embodiment includes NFC communication module 2161 and Bluetooth (registered trademark) communication module 2162. In practice, NFC communication module 2161 and Bluetooth communication module 2162 each include an antenna and a transmission/reception circuit that enable the respective wireless communication method.

NFC communication module 2161 can read out data written in the memory of wireless tag 30 provided in cuff 20′ and can write data in the memory of wireless tag 30 by performing wireless communication with wireless tag 30 in a wireless communication method conforming to an NFC standard.

Bluetooth communication module 2162 can wirelessly receive a control signal from blood pressure pulse wave analyzing apparatus 200 (FIG. 5) and wirelessly transmit a blood pressure pulse wave measurement result to blood pressure pulse wave analyzing apparatus 200 by performing wireless communication with blood pressure pulse wave analyzing apparatus 200 in a wireless communication method conforming to a Bluetooth standard.

In the present embodiment, although a case has been described as an example in which the communication with the wireless tag is performed by NFC and the communication with the blood pressure pulse wave analyzing apparatus is performed using Bluetooth, the present embodiment is not limited thereto. In short, it is sufficient that wireless communication unit 100′ includes the following: a first wireless section that performs wireless communication with wireless tag 30 provided in cuff 20′ using a first wireless communication method; and a second wireless section that performs wireless communication with an external apparatus (blood pressure pulse wave analyzing apparatus 200 in the embodiment) using a second wireless communication method that allows communication over a longer distance than the first wireless communication method. In addition, it is sufficient that wireless communication unit 100′ has a configuration such that wireless communication unit 100′ can perform wireless communication with wireless tag 30 and the external apparatus (blood pressure pulse wave analyzing apparatus 200) by using the first and second wireless sections.

Next, the blood pressure pulse wave examination using wireless cuff device 10′ will be described.

Before the blood pressure pulse wave examination, wireless tag 30 is provided in each cuff 20′ in advance. Each wireless tag 30 stores the information on the mounting site and the size of attached cuff 20′.

It is preferable that wireless tag 30 is built into cuff 20′ so as not to be removed by the user. As a result, it is possible to prevent a correspondence relationship between wireless tag 30 and cuff 20′ from being unintentionally disrupted.

The user, such as a medical worker, attaches wireless communication unit 100′ to each cuff 20′. As a result, as illustrated in FIG. 2, four wireless cuff devices 10-1′ to 10-4′ are prepared before the examination. As a matter of fact, wireless communication unit 100′ is attached to or detached from cuff 20′, for example, in the following scenes:—when wireless communication unit 100′ is attached to newly manufactured cuff 20′;—when cuff 20′ is deteriorated over time and is replaced;—when the size of cuff 20′ is changed at the examination site; and -when cuff 20′ is washed or disinfected.

Wireless communication units 100′ to be attached to cuffs 20′ have a common configuration regardless of the mounting site or the size of cuffs 20′. As a result, it is not necessary to prepare wireless communication unit 100′ as many as the number of cuffs 20′. For example, when certain cuff 20′ is washed or disinfected, wireless communication unit 100′ can be detached from cuff 20′ and detached wireless communication unit 100′ can be attached to another cuff 20′.

Next, the user mounts four wireless cuff devices 10-1′ to 10-4′ on the predetermined sites of the subject as illustrated in FIG. 3 (suffice it to say that 10-1 to 10-4 in FIG. 3 are replaced with 10-1′ to 10-4′, respectively). At this time, the user mounts wireless cuff devices 10-1′ to 10-4′ on the predetermined sites of the subject with the colors or the characters of edges 20a of cuffs 20-1′ to 20-4′ as a guide.

Next, the user turns on the power of wireless cuff devices 10-1′ to 10-4′ by operating power button 131. Wireless cuff devices 10-1′ to 10-4′ may be mounted on the subject after the power of wireless cuff devices 10-1′ to 10-4′ is turned on.

When the power of wireless cuff devices 10-1′ to 10-4′ is turned on and a predetermined operation is performed on blood pressure pulse wave analyzing apparatus 200 and/or wireless cuff devices 10-1′ to 10-4′ by the user, blood pressure pulse wave analyzing apparatus 200 and wireless cuff devices 10-1′ to 10-4′ are synchronized with each other and enter a state in which the examination can be performed.

At the start of the examination or before the start of the examination, wireless cuff devices 10-1′ to 10-4′ read out the information (the mounting sites, the sizes of the cuffs, and the types of the cuffs) in wireless tags 30-1 to 30-4 via NFC communication module 2161, and transmit the readout information to blood pressure pulse wave analyzing apparatus 200 via Bluetooth communication module 2162. As a result, blood pressure pulse wave analyzing apparatus 200 can recognize the mounting sites, the cuff sizes, and the types of the cuffs of wireless cuff devices 10-1′ to 10-4′, respectively.

When the user performs an operation of starting the blood pressure pulse wave examination, blood pressure pulse wave analyzing apparatus 200 transmits a control signal corresponding to the mounting site, the cuff size, and the type of the cuff to each of wireless cuff devices 10-1′ to 10-4′ using Bluetooth communication. In practice, the blood pressure pulse wave analyzing apparatus controls the cuff pressure in accordance with the cuff size and the type of the cuff.

Wireless cuff devices 10-1′ to 10-4′ control cuff drivers 140 based on a control signal from blood pressure pulse wave analyzing apparatus 200. As a result, the air is supplied to and discharged from the air bag of cuff 20′, and the cuff pressure is controlled to a value suitable for the examination.

Wireless cuff devices 10-1′ to 10-4′ transmit the cuff pressure detected by cuff pressure detector 150, namely the blood pressure pulse wave data, to blood pressure pulse wave analyzing apparatus 200 using Bluetooth communication. At this time, each of wireless cuff devices 10-1′ to 10-4′ (wireless communication units 100-1′ to 100-4′) transmits the blood pressure pulse wave data together with the information on the mounting site read out from corresponding one of wireless tags 30-1 to 30-4.

As a result, blood pressure pulse wave analyzing apparatus 200 can recognize which blood pressure pulse wave data is the data of which mounting site, for the received blood pressure pulse wave data.

Blood pressure pulse wave analyzing apparatus 200 calculates the indicator of the arterial stiffness, such as the pulse wave propagation velocity, by using the blood pressure pulse wave data of each mounting site. Various known processing can be applied to the calculation of the pulse wave propagation velocity or the like using the blood pressure pulse wave, and therefore, the description thereof will be omitted herein.

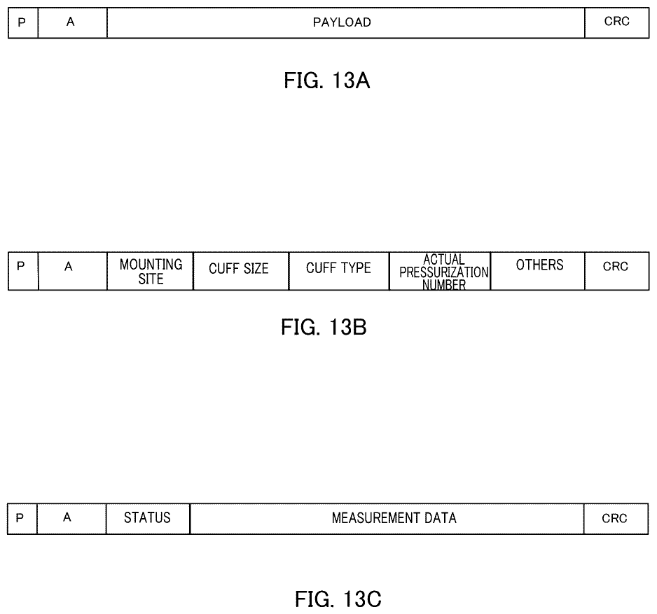

FIGS. 13A to 13C illustrate a format example of a packet transmitted from wireless communicator 160′ of wireless communication unit 100′ to blood pressure pulse wave analyzing apparatus 200. As illustrated in FIG. 13A, one packet is configured with a preamble (P), an address (A), a payload (PAYLOAD), and a checksum (CRC).

The preamble (P) is, for example, 1 byte. The address (A) is a unique number assigned to each wireless communication unit 100′, and is, for example, 5 bytes. The payload (PAYLOAD) is, for example, 32 bytes. The checksum (CRC) is, for example, 2 bytes.

FIG. 13B illustrates the content of the packet transmitted from wireless communication unit 100′ to blood pressure pulse wave analyzing apparatus 200 before the transmission of the measurement data (blood pressure pulse wave data) (for example, in a case of pairing). The payload (PAYLOAD) contains mounting site information, cuff size information, actual pressurization number information, and other information. The mounting site information is the right upper arm, the left upper arm, the right wrist, the left wrist, or the like. The cuff size information is S, M, L, or the like. The cuff type information is for an adult, for a child, for an infant, or the like. The actual pressurization number is the number of times the pressure in cuff 20′ is increased by cuff driver 140, and is, for example, 10, which will be described in detail below. The other information is the version number of software of wireless communication unit 100′ or the like.

FIG. 13C illustrates the content of the packet when the measurement data (blood pressure pulse wave data) is transmitted after the packet illustrated in FIG. 13B is transmitted. The payload (PAYLOAD) contains status information and the measurement data (blood pressure pulse wave data). The status information is information indicating the operation state of wireless communication unit 100′.

As described above, wireless cuff device 10′ of the present embodiment includes the following: cuff 20′ that includes an NFC tag (wireless tag 30) having the mounting site information; and wireless communication unit 100′ that is detachably attached to cuff 20′, is wirelessly connected to the NFC tag (wireless tag 30), and wirelessly transmits the mounting site information and data (blood pressure pulse wave data) obtained by driving cuff 20′ to the analyzing apparatus (blood pressure pulse wave analyzing apparatus 200).

From another viewpoint, wireless cuff device 10′ of the present embodiment includes the following: cuff 20′ that includes an air bag and is configured to be wound around the predetermined site of a subject; cuff driver 140 that supplies and discharges an air to and from the air bag of cuff 20′; cuff pressure detector 150 that detects the cuff pressure of cuff 20′ during the examination; and wireless communication unit 100′ that is detachably attached to cuff 20′. Wireless communication unit 100′ includes a first wireless section (in the embodiment, NFC communication module 2161) that performs wireless communication with wireless tag 30 provided in cuff 20′, and a second wireless section (in the embodiment, Bluetooth communication module 2162) that performs wireless communication with an external wireless communication unit.

As a result, it is possible to achieve wireless cuff device 10′ and the blood pressure pulse wave analyzing system that can achieve the hoseless configuration.

According to the configuration of the present embodiment, for example, the following effects can be obtained.
(i) Even when the user does not associate the mounting sites between wireless cuff devices 10-1′ to 10-4′ and blood pressure pulse wave analyzing apparatus 200, the association for the mounting sites between wireless cuff devices 10-1′ to 10-4′ and blood pressure pulse wave analyzing apparatus 200 is automatically performed by transmitting the information on the mounting sites stored in wireless tags 30-1 to 30-4 via wireless cuff devices 10-1′ to 10-4′, respectively. As a result, it is possible to reduce the workload of the user. In addition, it is possible to prevent incorrect association. That is, in the blood pressure pulse wave analyzing system using an air hose in the related art, when the user incorrectly associates the air hose with a connector of the blood pressure pulse wave analyzing apparatus (that is, when the user incorrectly connects the air hose with the connector), it is not possible to perform a correct examination. The configuration of the present embodiment can prevent such incorrect connection.
(ii) Wireless communication units 100 are each configured to be detachably attached to cuff 20′ and each wireless communication unit 100′ reads the information on the type of cuff 20′ from wireless tag 30 that is fixed and attached to cuff 20′. Therefore, wireless communication units 100′ can have a common configuration regardless of the mounting site or the size of cuff 20′. As a result, the user does not have to associate wireless communication unit 100′ with cuff 20′, and the general versatility of wireless communication unit 100′ can be further increased.

Here, unlike the above-described embodiment, when it is assumed that only wireless communication unit 100′ and blood pressure pulse wave analyzing apparatus 200 are wirelessly connected to each other without using wireless tag 30, the following disadvantages occur.

As a first method that does not use wireless tag 30, it is possible to use a wireless communication unit 100′ that is dedicated to each of the left upper arm, right upper arm, left lower leg, and right lower leg, rather than using common wireless communication units 100′ for all the mounting sites. Specifically, a wireless communication unit for the left upper arm is attached to the cuff of the left upper arm, a wireless communication unit for the right upper arm is attached to the cuff of the right upper arm, a wireless communication unit for the left lower limb is attached to the cuff of the left lower limb, and a wireless communication unit for the right lower limb is attached to the cuff of the right lower limb.

In addition, as a second method that does not use wireless tag 30, the following method is possible: the user manually sets wireless communication units 100′ to set cuff 20′ at which mounting site corresponds to which wireless communication unit 100′.

However, there is a possibility of attachment mistake in the first method and there is a possibility of incorrect setting in the second method. When such an attachment mistake or incorrect setting is made, the association between the detection part and the detected blood pressure pulse wave is incorrect in blood pressure pulse wave analyzing apparatus 200, so that it is not possible to obtain the correct examination result in blood pressure pulse wave analyzing apparatus 200.

Wireless tag 30 is used in the present embodiment, and therefore, the correct examination result can be obtained in blood pressure pulse wave analyzing apparatus 200 as long as the mounting site of cuff 20′ is not incorrect.
(iii) When the electric charge remaining in battery 110 of wireless communication unit 100′ is decreased, another wireless communication unit 100′ in which battery 110 is charged may be attached to cuff 20′. Newly attached wireless communication unit 100′ reads out the information on wireless tag 30 of cuff 20′ and transmits the readout information to blood pressure pulse wave analyzing apparatus 200. As a result, it is not necessary to set the correspondence relationship between wireless communication unit 100′ and cuff 20′ manually, so that wireless communication unit 100′ is easily replaced.
(iv) The communication method performed with wireless tag 30 is a communication method having a shorter communication distance than the communication method performed with blood pressure pulse wave analyzing apparatus 200, and therefore, the consumption of battery 110 of wireless communication unit 100′ is reduced, and the charging interval of battery 110 is prolonged.

### <2-2> Another Form in Embodiment 2

<2-2-1> Embodiment 2 describes the case in which the information on the mounting site and the size of cuff 20′ is stored in wireless tag 30, however, the present disclosure is not limited thereto. Any information can be used as long as the mounting site or the size of cuff 20′ can be specified on blood pressure pulse wave analyzing apparatus 200 side, and therefore, the stored information may be any information other than the information on the mounting site or the size itself.

<2-2-2> In addition to the configuration of Embodiment 2 described above, the following configuration is possible: the storage of wireless tag 30 stores a limit pressurization number that is a design durability pressurization number of cuff 20′; wireless communication unit 100′ writes, into the storage of wireless tag 30, an actual pressurization number that is the number of times the pressure in cuff 20′ is increased by cuff driver 140; and when the actual pressurization number becomes equal to or greater than the limit pressurization number stored in advance, an alarm is output indicating that the actual pressurization number becomes equal to or greater than the limit pressurization number.

Specifically, the description therefor will be made. The limit pressurization number stored in advance in wireless tag 30 is, for example, 30,000. Incidentally, the limit pressurization number in the cuff for a toe is, for example, about 1,000. Wireless tag 30 has a memory region for storing the actual pressurization number. Wireless communication unit 100′ increases the actual pressurization number stored in wireless tag 30 in increments each time the pressure in cuff 20′ is increased. Wireless communication unit 100′ reads out the limit pressurization number and the actual pressurization number recorded in wireless tag 30, compares the limit pressurization number with the actual pressurization number, and prompts replacement of cuff 20′ by an alarm output by display or sound. For example, wireless communication unit 100′ outputs the alarm when the actual pressurization number becomes equal to or greater than the limit pressurization number.

In Embodiment 2, since the pressurization numbers including the actual pressurization number is recorded in wireless tag 30 that is fixed and attached to cuff 20′, it is possible to, for example, change wireless communication unit 100′ conveniently. That is, since cuff 20′ itself has the information on the pressurization numbers, the pressurization numbers are carried over without depending on the replacement of wireless communication unit 100′.

<2-2-3> Embodiment 2 describes the case in which the blood pressure pulse wave data is transmitted together with the information on the mounting sites read out from wireless tags 30-1 to 30-4 via wireless cuff devices 10-1′ to 10-4′ (wireless communication units 100-1′ to 100-4′), however, the present disclosure is not limited thereto. Blood pressure pulse wave analyzing apparatus 200 only needs to specify which one of wireless cuff device 10-1′ to 10-4′ (wireless communication unit 100-1′ to 100-4′) transmits the blood pressure pulse wave data of which mounting site. Therefore, for example, each of wireless cuff devices 10-1′ to 10-4′ (wireless communication units 100-1′ to 100-4′) may transmit the information for specifying the mounting site to blood pressure pulse wave analyzing apparatus 200 only once, and blood pressure pulse wave analyzing apparatus 200 may associate the identification information (ID) of each of wireless cuff devices 10-1′ to 10-4′ (wireless communication units 100-1′ to 100-4′) with the corresponding mounting site. Then, thereafter, when each of wireless cuff devices 10-1′ to 10-4′ (wireless communication units 100-1′ to 100-4′) transmits the blood pressure pulse wave data together with the ID of the own device, blood pressure pulse wave analyzing apparatus 200 can recognize the relationship between the blood pressure pulse wave data and the mounting site via the ID.

<2-2-4> In Embodiment 2 described above, it is possible that the user unintentionally attaches wrong cuff 20′. For example, it is assumed that wireless tag 30-1 for the left upper arm is incorrectly attached to cuff 20-2′ for the right upper arm. In this case, blood pressure pulse wave analyzing apparatus 200 receives the information on the mounting sites indicating that the information is on the left upper arm from two wireless communication units 100-1′ and 100-2′. In this case, blood pressure pulse wave analyzing apparatus 200 may output an alarm indicating that wrong wireless tag 30 is attached to cuff 20′.

Wireless communication units 100-1′ to 100-4′ may be dedicated to the left upper arm, the right upper arm, the left lower limb, and the right lower limb, respectively, with regard to the mounting sites, and an error may be output from wireless communication units 100-1′ to 100-4′ when it is determined that at least one of wireless communication units 100-1′ to 100-4′ is attached to wrong cuff 20′ with reference to the information on the mounting sites from wireless tags 30-1 to 30-4.

<2-2-5> Embodiment 2 describe the case in which blood pressure pulse wave analyzing apparatus 200 examines the state of a blood vessel by using a blood pressure pulse wave detected by each of wireless cuff devices 10-1′ to 10-4′, however, blood pressure pulse wave analyzing apparatus 200 may examine the state of the blood vessel by using the heart sound in addition to the blood pressure pulse wave. In this case, the blood pressure pulse wave analyzing system may be configured to include a heart sound sensor in addition to the configuration of FIG. 3. Here, blood pressure pulse wave analyzing apparatus 200 and the heart sound sensor are configured to be wirelessly connected to each other by, for example, Bluetooth communication. The examination of the blood vessel using the blood pressure pulse wave and the heart sound is a known technique described in, for example, PTL 1, and thus the description thereof will be omitted herein.

<2-2-6> Embodiment 2 describes the case in which the wireless cuff device according to the present disclosure is used in the blood pressure pulse wave analyzing system, however, the wireless cuff device according to the present disclosure is not limited thereto, and can be applied to a system other than the blood pressure pulse wave analyzing system. That is, the above-described embodiment describes the case in which wireless communication unit 100′ transmits the examination result to blood pressure pulse wave analyzing apparatus 200, however, the external apparatus with which wireless communication unit 100′ cooperates and to which wireless communication unit 100′ transmits the examination result is not limited to blood pressure pulse wave analyzing apparatus 200.

As the external apparatus with which wireless communication unit 100′ cooperates and to which wireless communication unit 100′ transmits the examination result, for example, a biological information monitor, a Holter blood pressure monitor, exercise stress blood pressure monitor, or the like is possible. A control signal or the like is transmitted from the external apparatus to wireless communication unit 100′. Blood pressure value data, cuff mounting position information, and the like are transmitted from wireless communication unit 100′ to the external apparatus.

That is, as one aspect of the present disclosure, an examination system including the following is possible: wireless cuff device 10′; and an analyzing apparatus (for example, a biological information monitor, a Holter blood pressure monitor, exercise stress blood pressure monitor, or the like) that is provided with an external wireless section and wirelessly transmits at least a control signal for controlling cuff driver 140 to wireless cuff device 10′ and wirelessly receives the information on wireless tag 30 and the cuff pressure from wireless cuff device 10′.

<2-2-7> Embodiment 2 describes the case in which cuff driver 140 and cuff pressure detector 150 are provided in wireless communication unit 100′, however, cuff drive unit 140 and cuff pressure detector 150 may be provided in a unit separate from the wireless communication unit in which wireless communicator 160′ is provided. In this case, the separate unit may be configured to be detachably attached to cuff 20′ or may be fixed to cuff 20′. The wireless communication unit may be directly attached to cuff 20′ or may be indirectly attached to cuff 20′ via the separate unit. When the wireless communication unit is electrically connected to the separate unit, the same operation as in the above-described embodiment can be achieved.

<2-8> Conclusion of Embodiment 2 and Another Forms One aspect of a cuff device according to the present disclosure includes the following:
a cuff that includes an NFC tag having mounting site information; and
a wireless communication unit (that is detachably attached to the cuff) is wirelessly connected to the NFC tag and wirelessly transmits the mounting site information and data obtained by driving the cuff to an analyzing apparatus.

In one aspect of the wireless cuff device according to the present disclosure,
the wireless communication unit wirelessly transmits information on the cuff to the analyzing apparatus prior to the transmission of the data, the information including the mounting site information.

In one aspect of the wireless cuff device according to the present disclosure,
a single packet wirelessly transmitted by the wireless communication unit to the analyzing apparatus prior to the transmission of the data includes at least one of size information of the cuff and type information of the cuff in addition to the mounting site information.

In one aspect of the wireless cuff device according to the present disclosure,
the cuff includes an air bag and is configured to be wound around a predetermined site of a subject; and
the wireless communication unit further includes a cuff driver that supplies and discharges air to and from the air bag of the cuff, and a cuff pressure detector that detects a cuff pressure of the cuff during an examination.

In one aspect of the wireless cuff device according to the present disclosure,
the cuff driver and the cuff pressure detector are provided in the wireless communication unit.

In one aspect of the wireless cuff device according to the present disclosure,
the analyzing apparatus is a blood pressure pulse wave analyzing apparatus; and
the wireless communication unit at least wirelessly receives, from the blood pressure pulse wave analyzing apparatus, a control signal for controlling the cuff driver, and wirelessly transmits, to the blood pressure pulse wave analyzing apparatus, the mounting site information and data on the cuff pressure obtained by driving the cuff.

In one aspect of the wireless cuff device according to the present disclosure,
the NFC tag includes a storage, and the storage stores information indicating a size and/or a type of the cuff in addition to the mounting site information.

In one aspect of the wireless cuff device according to the present disclosure,
the NFC tag includes a storage, and the storage stores a limit pressurization number of the cuff; and
the wireless communication unit writes, into the storage, an actual pressurization number that is the number of times pressure in the cuff is increased by the cuff driver, and when the actual pressurization number becomes equal to or greater than the limit pressurization number, the wireless communication unit outputs an alarm indicating that the actual pressurization number becomes equal to or greater than the limit pressurization number.

One aspect of an examination system according to the present disclosure includes the following:
the wireless cuff device; and
an analyzing apparatus that wirelessly receives, from the wireless cuff device, the mounting site information and data obtained by driving the cuff.

One aspect of an examination system according to the present disclosure includes the following:
the wireless cuff device; and
an analyzing apparatus that wirelessly transmits a control signal for controlling the cuff driver to the wireless cuff device and wirelessly receives the mounting site information and data obtained by driving the cuff from the wireless cuff device.

### <3> Embodiment 3

### <3-1> Knowledge Leading to Embodiment 3

Before describing Embodiment 3, knowledge of the inventors of the present disclosure leading to Embodiment 3 will be described.

In the related art, electronic apparatuses such as a personal computer perform time synchronization between apparatuses by using a time synchronization method such as a network time protocol (NTP).

In addition, in the related art, there are various algorithms such as a timing-sync protocol for sensor network (TPSN), a flooding synchronization time protocol (FTSP), a Tiny-Sync (it should be confirmed whether it is an abbreviation for “Tight time Synchronization”), and a reference broadcast synchronization (RBS) as representative time synchronization algorithms for time-synchronizing a wireless sensor network (WSN). These time synchronization algorithms are described in, for example, NPL 1.

TPSN is a method of performing time synchronization by measuring a radio wave propagation time. RBS is a method of synchronizing receivers instead of synchronizing a transmitter and a receiver. FTSP is a method of synchronizing time points of a transmitter with a plurality of receivers using a single packet. Tiny-Sync is an exact time synchronization method based on measurement of the radio wave propagation time.

Meanwhile, in NTP, an error of about 100 ms occurs due to network delays, processing delays of basic software (OS) and hardware, and the like.

In addition, in the wireless communication between a transmitter and a receiver, as described in NPL 2, a time synchronization error caused by a “non-deterministic” delay time that cannot be estimated in advance occurs, so that it is difficult to suppress the time synchronization error to a very small level. In consideration of the above points, Embodiment 3 of the present disclosure has been reached.

In a time synchronization method, there are the following methods paired with each other. In Embodiment 3, one of the following methods paired with each other is used.
Master & slave method: one node is set as a master, and a reference time of the master is synchronized with a slave node (TPSN and FTSP correspond to this method). Peer-to-peer method: nodes on a network directly communicate with all the nodes and exchange time point information (RBS corresponds to this method). In Embodiment 3, the master & slave method is adopted among the two methods.
Clock correction method: a clock of each node is corrected for each time synchronization processing.—Untethered clock method: a clock of each node is allowed to operate freely, and information for converting local times into each other's time points is exchanged and stored. In the present embodiment, the untethered clock method is adopted among the two methods.
Internal synchronization method: the clock error of each node is relatively minimized.—External synchronization method: the clock of each node is synchronized to a reference time point such as world standard time (UTC), for example (global positioning system (GPS) corresponds to this method). In Embodiment 3, the internal synchronization method is adopted among the two methods.
Sender-to-receiver method: time point information is transmitted from a transmitter of a node, and a receiver synchronizes with the transmitter based on the received information.—Receiver-to-receiver method: this method uses the characteristic that when two or more receivers receive the same message packet, they receive the packet almost simultaneously. In Embodiment 3, the sender-to-receiver method is adopted among the two methods.

In addition, in Embodiment 3, as the time synchronization method, a round trip time method or a time of flight (TOF) method, that is, a method of measuring the one-way radio wave propagation time from the time required for the radio wave to travel back and forth between the nodes is adopted.

Next, the time synchronization error will be described.

In order to achieve accurate time synchronization, it is necessary to eliminate non-determinism. In order to understand the cause of the error, a method of decomposing the delay time of a message into factors and analyzing the factors is proposed in, for example, NPL 1. The “non-determinism or non-deterministic” means that the delay time cannot be estimated in advance due to randomness (fluctuation). The “determinism or deterministic” means a delay time that can be estimated by theoretical calculation.

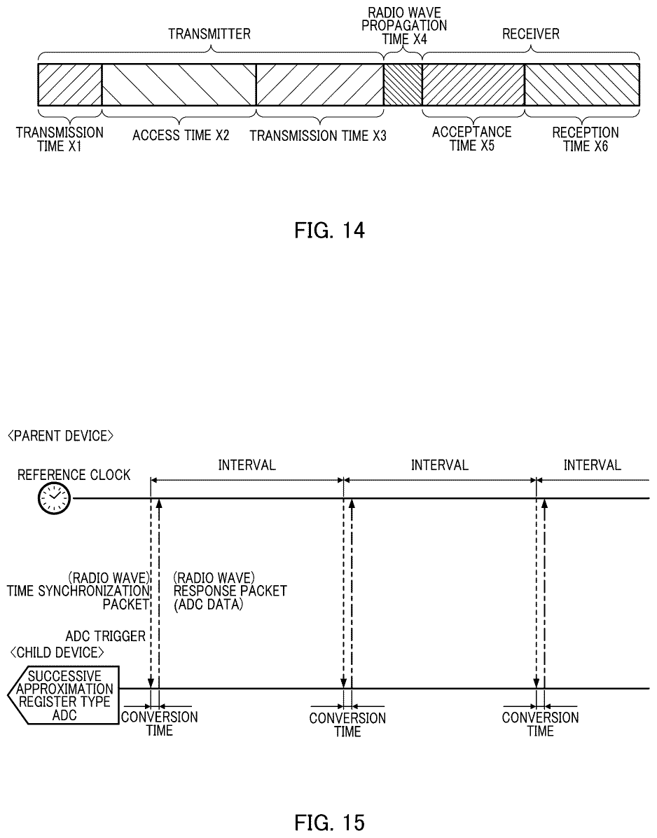

FIG. 14 illustrates factors of the delay time of a message from a wireless transmitter to a wireless receiver. The left end in the drawing indicates a transmission start time point, and the right end in the drawing indicates a reception end time point.

Transmission time X1 is a time spent on creating a message packet by a transmission-side host computer (hereinafter, a host computer is simply referred to as a host). Transmission time X1 includes a transmission time from the host to the transmitter. In other words, transmission time X1 is a waiting time for process/task execution of an operating system, and is non-deterministic.

Access time X2 is a delay that occurs while waiting for access to a transmission channel. In other words, access time X2 is a waiting time until another transmitter ends its transmission to the same channel, and is non-deterministic.

Wave transmission time X3 is a time required for the transmitter to transmit the message one bit at a time. Transmission time X3 can be estimated from the length of the message and a wireless communication speed and is deterministic.

Radio wave propagation time X4 is a propagation time of radio waves from the transmitter to the receiver. Radio wave propagation time X4 depends on the fact that radio waves travel 300,000 km in one second and take about 3.3 ns to travel one meter and is deterministic.

Acceptance time X5 is a time required for the receiver to receive a message one bit at a time. Acceptance time X5 can be estimated from the length of the message and a wireless communication speed and is deterministic.

Reception time X6 is a time required for the message packet to reach a reception-side host. Reception time X6 includes a transmission time from the receiver to the host. In other words, reception time X6 is a waiting time for process/task execution of an operating system and a waiting time for an interrupt of a receiver driver and is non-deterministic.

As can be seen from the above, the non-deterministic factors are transmission time X1, access time X2, and reception time X6. In Embodiment 3, the non-determinism is eliminated for transmission time X1 and reception time X6 by performing real-time processing without an operating system. In addition, the non-determinism is eliminated for access time X2 by performing frequency hopping to avoid carrier sense required by technical standards approval (TSA).

Deterministic wave transmission time X3 and deterministic acceptance time X5 are estimated from the message length and the transmission rate. Radio wave propagation time X4 is estimated by measuring the round trip time.

Here, the inventors of the present invention consider applying the time synchronization method according to Embodiment 3 to a blood pressure pulse wave analyzing system or a medical apparatus that performs pulse wave measurement, heart sound detection, and electrocardiogram measurement. In these medical apparatuses, in order to obtain necessary resolution and accuracy, a 24-bit ΔΣ type AD converter is currently employed.

ΔΣ type AD converters have the advantages of, for example, having a high signal-to-noise ratio (SNR), being able to achieve a resolution of 20 bits or more, being easy to correct linearity errors, and not generating missing codes in principle. On the other hand, ΔΣ type AD converters have the disadvantages of, for example, having a relatively low sampling rate and being slow to start up, making the converters unsuitable for applications that require high-speed switching of the AD converter input.

Another AD converter that is different from the ΔΣ type AD converter is the successive approximation register (SAR) type AD converter. The successive approximation register type AD converter has advantages of, for example, being able to reduce a sampling frequency as much as desired to perform one-shot operation by a trigger, and being able to easily increase the number of input channels by using a multiplexer in combination. On the other hand, the successive approximation register type AD converter has disadvantages of, for example, deteriorating DNL characteristics and difficulty in obtaining high accuracy with a resolution of 18 bits or more, and generation of missing codes when accuracy is poor.

Currently, in order to obtain the resolution and the accuracy of a medical apparatus, it is preferable to use the ΔΣ type AD converter.

First, the time synchronization of a ΔΣ type AD converter is analyzed in comparison with a SAR type AD converter.

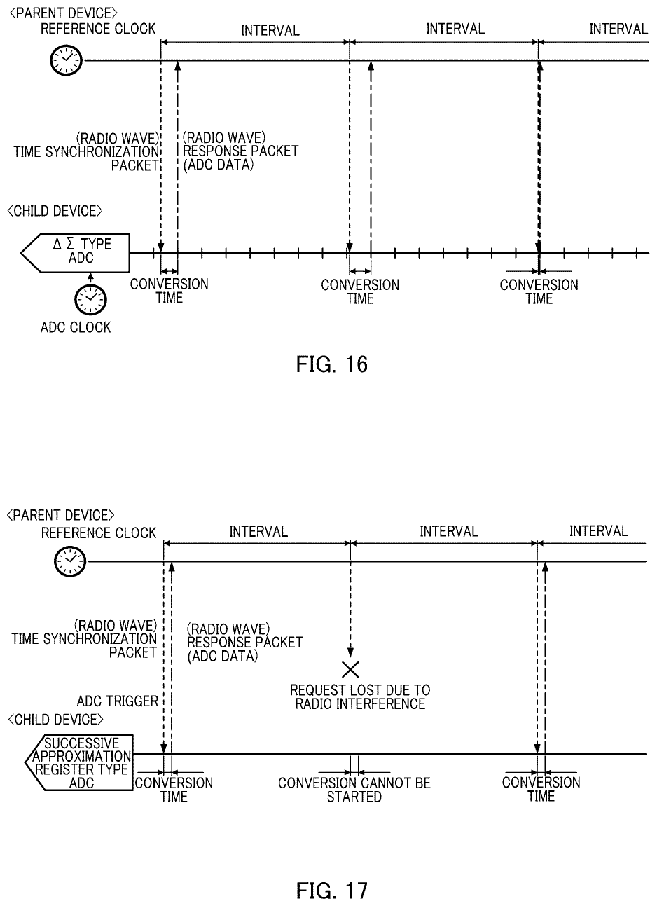

FIG. 15 is a diagram for describing time synchronization when a successive approximation register type AD converter (ADC) is used, and FIG. 16 is a diagram for describing time synchronization when a ΔΣ type AD converter (ADC) is used.

In these drawings, for example, the host device can be thought of as a medical apparatus main body equipped with a host computer, and the client device can be thought of as a sensor device that is mounted on a subject and measures the pulse wave, the electrocardiogram, or the like. Measurement data measured by the client device is digitally converted by the AD converter and transmitted to the host device. The host device calculates the pulse wave propagation velocity or the like based on the received measurement data.

In these drawings, the AD conversion is performed once in one interval for simplification, however, it is also possible to perform the AD conversion a plurality of times by using a time synchronization request as a trigger. For example, when an interval period is 100 ms and an AD conversion period is 1 ms, 100 samples of the measurement data are returned in one interval.

In a system using a successive approximation register type AD converter illustrated in FIG. 15, it is not difficult to construct a system in which the AD conversion is performed at constant time intervals with a time synchronization packet as a trigger and the measurement data is loaded into a response packet and returned. Although a constant time is required for a conversion time of the successive approximation register type ADC, the conversion time is a “deterministic” time delay, and thus does not lead to an error in the time synchronization.

On the other hand, in a system using a ΔΣ type AD converter illustrated in FIG. 16, the ΔΣ type AD converter performs AD conversion in synchronization with a clock for ADC, a time from the time synchronization request to the AD conversion completion is a non-deterministic time delay, and therefore, the performance of the time synchronization deteriorates. Even when the AD conversion is started after synchronizing a transmission-side reference clock and an ADC clock, the phases will eventually deviate from each other. When the ADC clock is corrected (skew adjustment) during the AD conversion, the period will be disrupted and the accuracy of the AD conversion will be deteriorated.

In the method of Embodiment 3, the problem of the untethered clock is solved.

As another problem, as illustrated in FIG. 17, when a wireless radio interference occurs and the time synchronization packet is lost, an AD conversion trigger cannot be set, and one interval's worth of measurement data may be lost. This is a problem unique to wireless technology, and occurs in both cases where a successive approximation register type AD converter is used and where a ΔΣ type AD converter is used. It is also possible to perform an operation in which the client device automatically performs the AD conversion when a time synchronization request does not arrive; however, an error relative to the reference clock still occurs on the transmission side for a waiting time required to confirm that the time synchronization request does not “arrive”. Alternatively, it may be possible for the transmission side to detect the radio interference to retransmit the time synchronization request, however, in this case, the interval is broken.

In the method of Embodiment 3, a method of maintaining accurate synchronization and a constant interval even when a radio interference occurs is presented.

### <3-2> Detailed Description of Embodiment 3

Hereinafter, Embodiment 3 will be described in detail with reference to the drawings.

### <3-2-1> Basic Processing

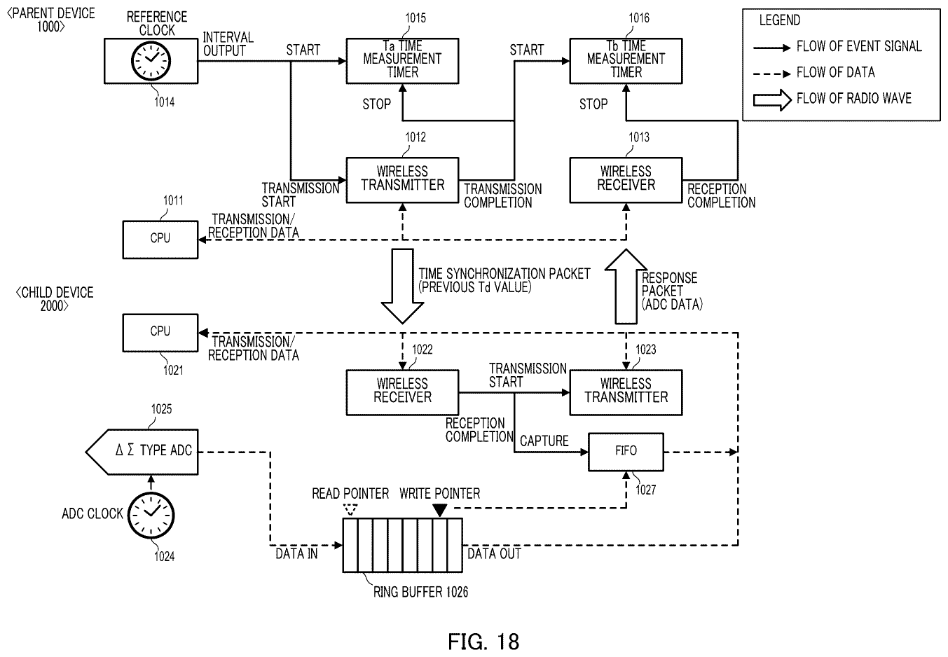

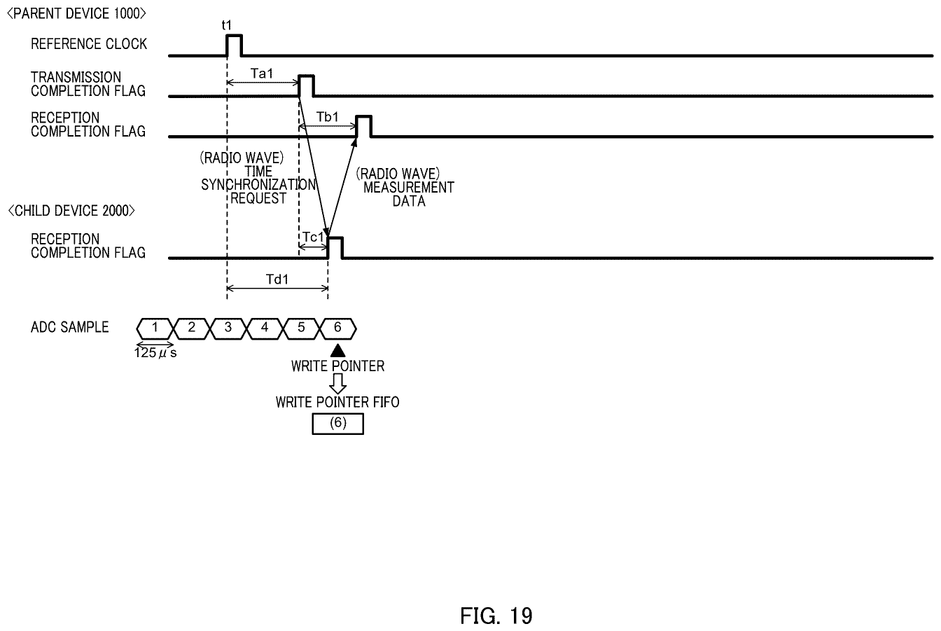

FIG. 18 is a block diagram for describing a time synchronization method according to Embodiment 3. Here, host device 1000 is, for example, an apparatus main body of the blood pressure pulse wave analyzing apparatus, and client device 2000 is, for example, a pulse wave sensor. The pulse wave sensor (client device 2000) transmits detected blood pressure pulse wave data to the apparatus main body (host device 1000) in a wireless manner, and the apparatus main body (host device 1000) calculates a pulse wave propagation velocity based on the blood pressure pulse wave data received from the pulse wave sensor (client device 2000). In practice, the apparatus main body (host device 1000) calculates the pulse wave propagation velocity based on the blood pressure pulse wave data received from a plurality of the pulse wave sensors (client devices 2000). Therefore, in order to accurately calculate the pulse wave propagation velocity, the apparatus main body (host device 1000) needs to perform time synchronization with the plurality of pulse wave sensors (client devices 2000) with high accuracy. Host device 1000 and client device 2000 do not necessarily have to be the apparatus main body of the blood pressure pulse wave analyzing apparatus and the pulse wave sensor. The method of Embodiment 3 can be widely applied to wireless apparatuses that require highly accurate time synchronization.

Host device 1000 includes central processing unit (CPU) 1011, wireless transmitter 1012, wireless receiver 1013, reference clock generator 1014, Ta time measurement timer 1015, and Tb time measurement timer 1016. CPU 1011, wireless transmitter 1012, and wireless receiver 1013 are connected by a data bus.

Client device 2000 includes CPU 1021, wireless transmitter 1022, wireless receiver 1023, ADC clock generator 1024, ΔΣ type AD converter 1025, ring buffer 1026, and first in, first out (FIFO) memory 1027. Hereinafter, the FIFO memory is abbreviated as FIFO. CPU 1021, wireless receiver 1022, wireless transmitter 1023, ΔΣ type AD converter 1025, ring buffer 1026, and FIFO 1027 are connected by a data bus.

FIG. 18 illustrates a flow of an event signal, a flow of data, a flow of radio waves, and the like.

The operations of host device 1000 and client device 2000 in FIG. 18 will be described with reference to FIGS. 19 to 32.

In host device 1000, when a reference clock is generated from reference clock generator 1014, the reference clock is output to wireless transmitter 1012 and Ta time measurement timer 1015. Wireless transmitter 1012 starts transmission at a timing at which the reference clock is input, and outputs a transmission completion signal to Ta time measurement timer 1015 and Tb time measurement timer 1016 when the transmission is completed. Ta time measurement timer 1015 measures time Ta from the reference clock to the transmission completion. Tb time measurement timer 1016 measures time Tb from the transmission completion to the reception completion.

In client device 2000, ΔΣ type AD converter 1025 performs the AD conversion based on the clock from ADC clock generator 1024. In the example of the present embodiment, as illustrated in FIGS. 19 to 23, the AD conversion is performed at a sampling frequency of 8 kHz (sampling period of 125 μs). For simplicity of the drawings, the number of samples of the AD conversion is roughly illustrated.

Radio wave propagation time Tc from host device 1000 to client device 2000 can be obtained by the equation of Tc=Tb/2. In addition, as can be seen from FIG. 19, time Td from a reference timing of the reference clock to the reception completion of wireless receiver 1022 of client device 2000 can be obtained by the equation of Td=Ta+Tc. The calculation of time Td is performed by a calculation unit such as CPU 1011.

FIG. 19 illustrates initial transmission from host device 1000 to client device 2000. In the initial transmission, a time synchronization request signal is transmitted from host device 1000 to client device 2000, and client device 2000 immediately transmits the measurement data to host device 1000 when the time synchronization request signal is received (when a reception completion flag is raised). In addition, in client device 2000, since the ADC sample when the time synchronization request signal is received is “6”, that is, the write pointer of ring buffer 1026 is “6”, the value “6” is put into the write pointer FIFO.

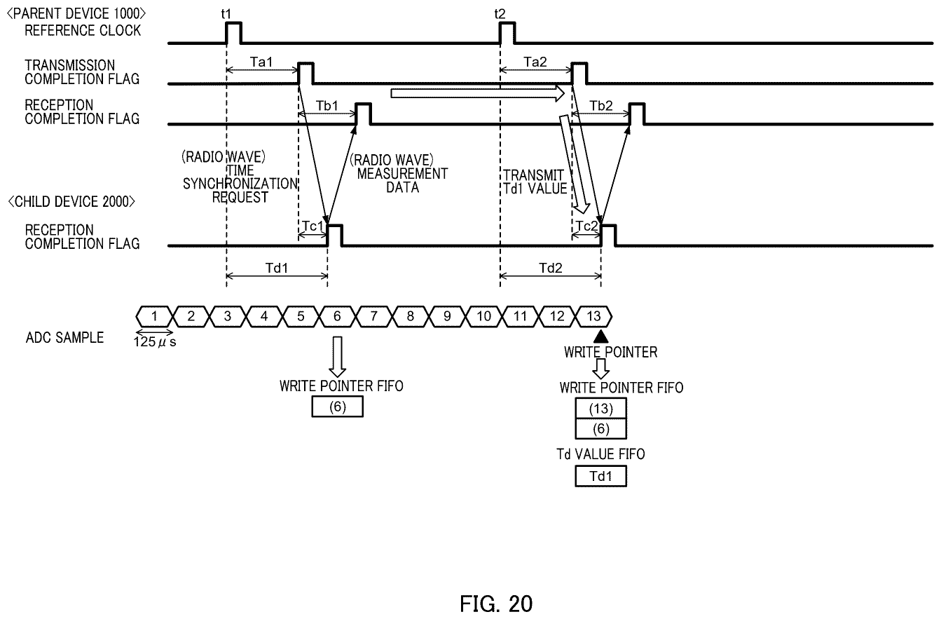

As illustrated in FIG. 20, when next reference clock t2 rises in host device 1000, host device 1000 performs the next transmission. At this time, host device 1000 sets, in the transmission data of wireless transmitter 1012, a value of Td1 calculated from Ta1, Tb1, and Tc1 in the previous transmission/reception and transmits the value to client device 2000. Client device 2000 puts the received value of Td1 into the Td value FIFO. In addition, since the ADC sample when Td1 is received is “13”, that is, the write pointer of ring buffer 1026 is “13”, the value “13” is put into the write pointer FIFO.

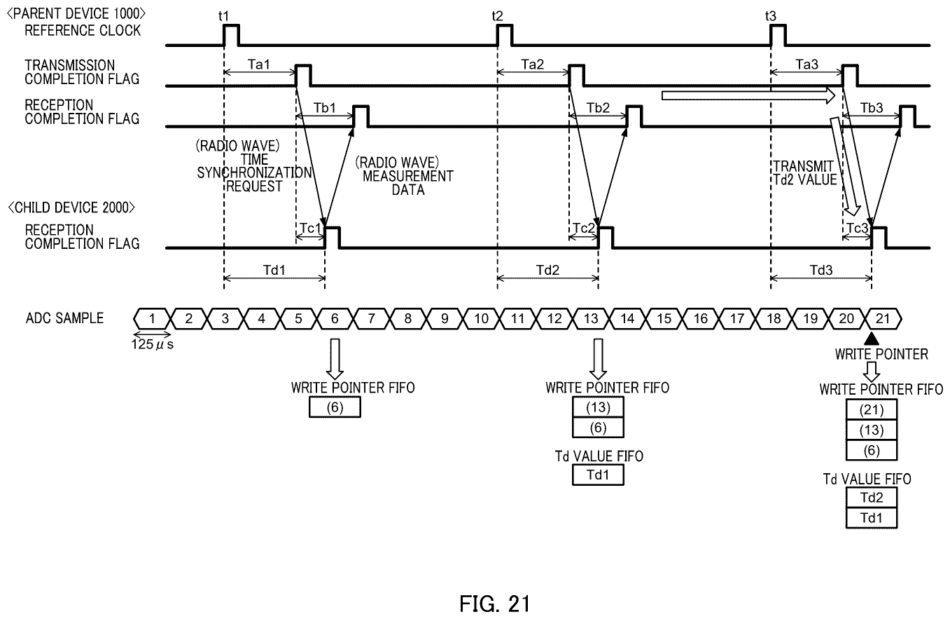

As illustrated in FIG. 21, when next reference clock t3 rises in host device 1000, host device 1000 performs the next transmission. At this time, host device 1000 sets, in the transmission data of wireless transmitter 1012, a value of Td2 calculated from Ta2, Tb2, and Tc2 in the previous transmission/reception and transmits the value to client device 2000. Client device 2000 puts the received value of Td2 into the Td value FIFO. In addition, since the ADC sample when Td2 is received is “21”, that is, the write pointer of ring buffer 1026 is “21”, the value “21” is put into the write pointer FIFO.

As described above, in Embodiment 3, host device 1000 transmits the calculated Td value to client device 2000 in the next reference clock (may be referred to as the next transmission cycle). In this manner, it is not necessary to provide dedicated hardware.

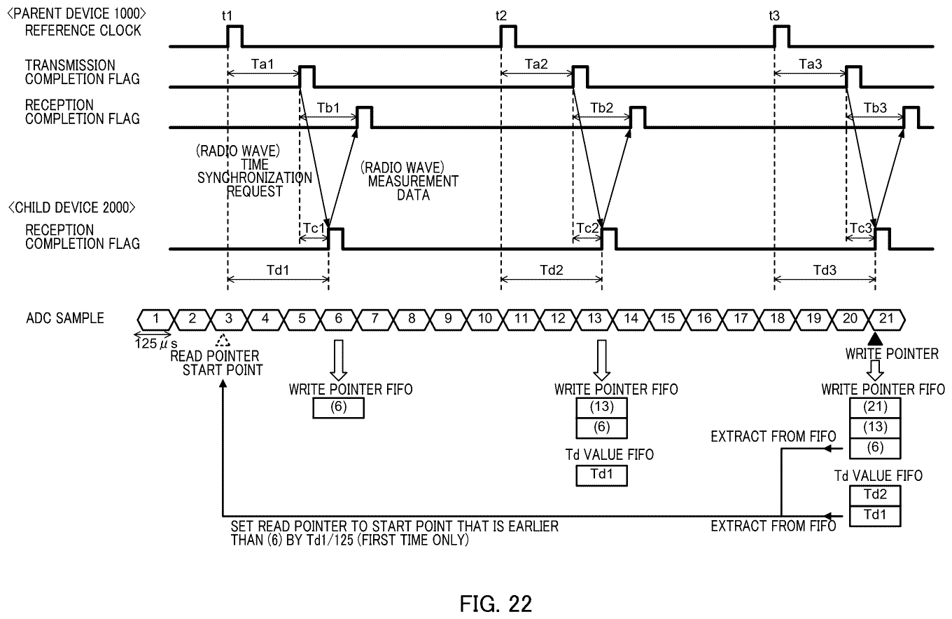

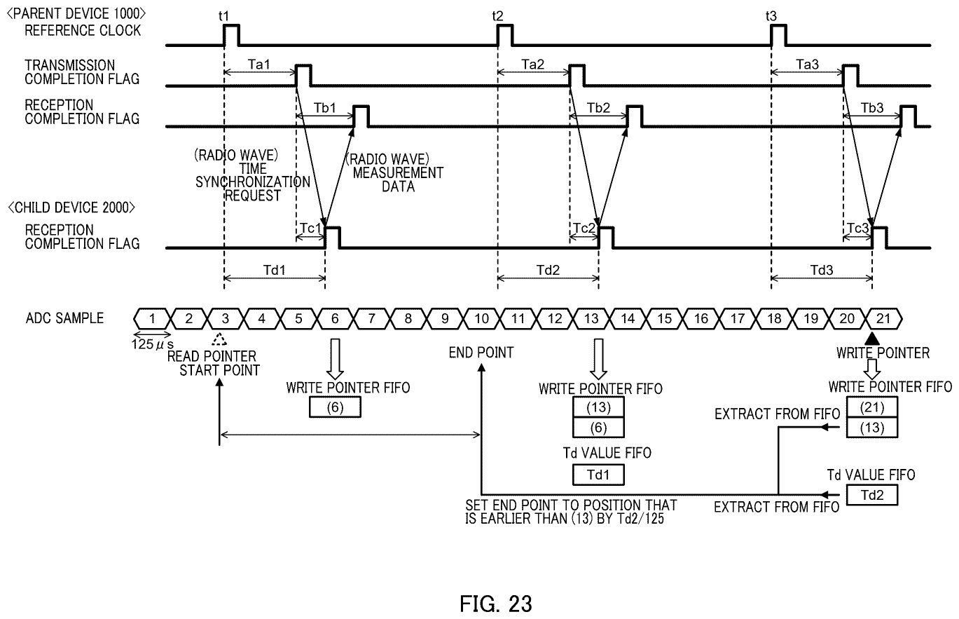

As illustrated in FIGS. 22 and 23, client device 2000 sets the start point and the end point of the read pointer based on the Td value.

First, as illustrated in FIG. 22, client device 2000 extracts the write pointer value “6” and Td1 stored in FIFO 1027, and sets the start point to a position that is earlier than the write pointer value “6” by N=Td1/125 [pieces] (an example when the unit of Td is μs and the sampling frequency of ΔΣ type AD converter 1025 is 8 kHz). The position of the start point matches the rising position t1 of the reference clock of host device 1000.

Next, as illustrated in FIG. 23, when the Td value is received, client device 2000 extracts the write pointer value “13” and Td2 stored in FIFO 1027, sets the end point to a position that is earlier than the write pointer value “13” by N=Td2/125 [pieces], and advances the read pointer while extracting the ADC data of ring buffer 1026 from the current position of the read pointer to the end point. As described above, client device 2000 sets the ADC data that is earlier than the position of the write pointer stored in FIFO 1027 by the result of dividing the time Td by the sampling period of ΔΣ type AD converter 1025 as the end point of the readout position. Here, the term “result of dividing the time Td by the sampling period of ΔΣ type AD converter 1025” includes a result obtained by rounding the result of dividing the time Td by the sampling period of ΔΣ type AD converter 1025, a result obtained by truncating the result of dividing the time Td by the sampling period of ΔΣ type AD converter 1025, and the like.

The extracted data serves as the ADC data of the previous section synchronized with the reference clock, and therefore, the extracted data is set in wireless transmitter 1022 and transmitted to host device 1000 at a response timing for next reference clock t4.

In Embodiment 3, the ADC sample data is sequentially transmitted while the same processing as described above is carried out with a delay of two clocks from the reference clock of host device 1000.

As described above, in Embodiment 3, host device 1000 measures time Ta that is a time from the reference clock to the transmission of the signal to client device 2000 and time Tb that is a time from the transmission of the signal to the reception of the response signal from client device 2000, calculates time Td that is a time from the reference clock to the reception of the signal by client device 2000 based on time Ta and time tb, and transmits time Td to client device 2000. Client device 2000 transmits the data in the range corresponding to time Td.

Specifically, client device 2000 calculates the timing of the reference clock of host device 1000 from time Td and transmits the data in the range corresponding to the interval of the reference clock.

As a result, client device 2000 can wirelessly transmit the ADC data synchronized with the reference clock of host device 1000 without synchronizing ΔΣ type AD converter 1025 (that is, in the untethered clock state).

Incidentally, in the above-described processing of setting the start point to a position that is earlier by N=Td1/125 [pieces], when the division Tdx/125 does not result in an integer, a calculation result including a decimal fraction will be obtained. Since N is an integer, in normal software implementation, the decimal fraction is truncated or rounded. However, depending on the phase difference between the clock of host device 1000 and the clock of client device 2000, a deviation of less than one sample may occur even when either the truncation processing or the rounding processing is performed.

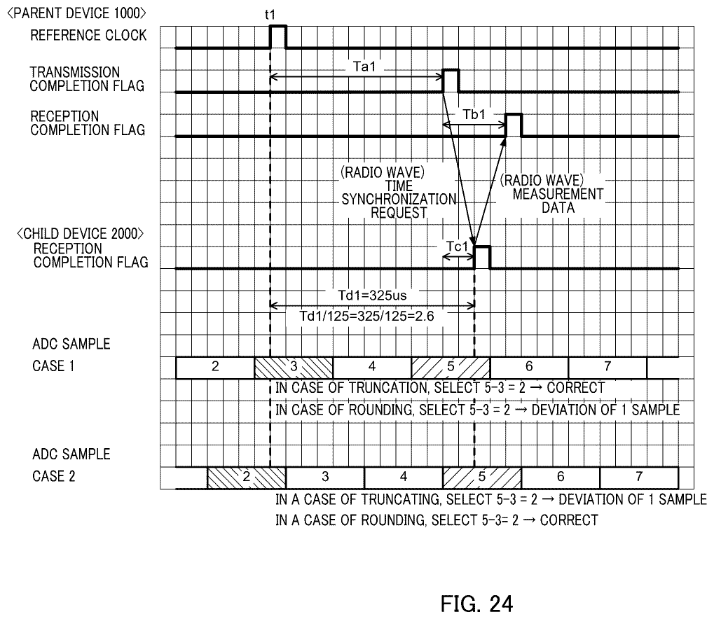

FIG. 24 illustrates the state thereof. It should be noted that 1 square in FIG. 24 represents 25 us. Here, it is assumed that the ADC sampling rate is 8 kHz (125 us), and Td1=325 us is measured by a timer. As a result of the calculation, N=Td1/125=2.6 is satisfied.

When a phase difference between the reference clock and the ADC clock is a case 1 in the drawing, when 2.6 is subjected to the truncation processing and going back two samples from “5” received as Tc1 results in “3”, which matches the timing of t1, and when 2.6 is rounded (rounded up) and going back three samples results in “2”, which is one sample away from the timing of t1.

However, when the phase difference between the reference clock and the ADC clock is a case 2 in the drawing, when 2.6 is subjected to the truncation processing and going back two samples from “5” received as Tc1 results in “3”, which is one sample away from the timing of t1, and when 2.6 is rounded (rounded up) and going back three samples results in “2”, which matches the timing of t1.

As described above, even when the remainder of the division of N=Tdx/125 is processed in any manner, the deviation of less than one sample may occur depending on the phase difference between the clock of host device 1000 and the clock of client device 2000. However, since the ADC data is finally oversampled, the deviation is within an allowable range, and the system may be implemented as it is.

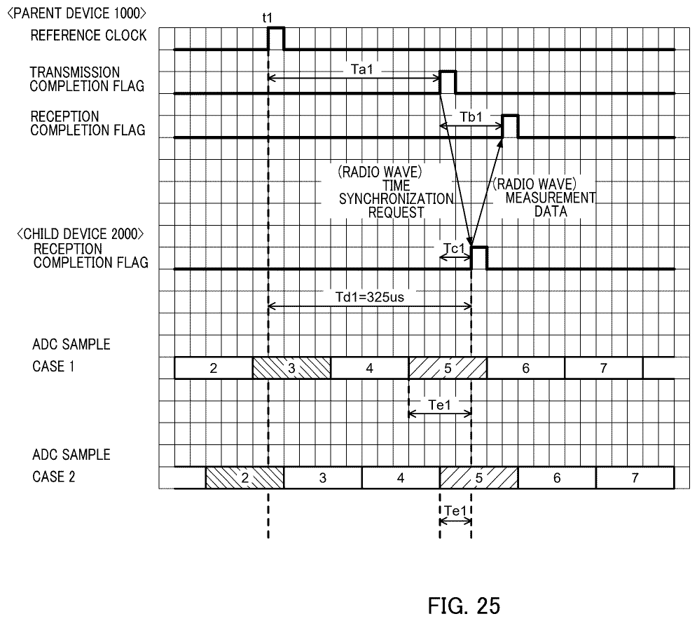

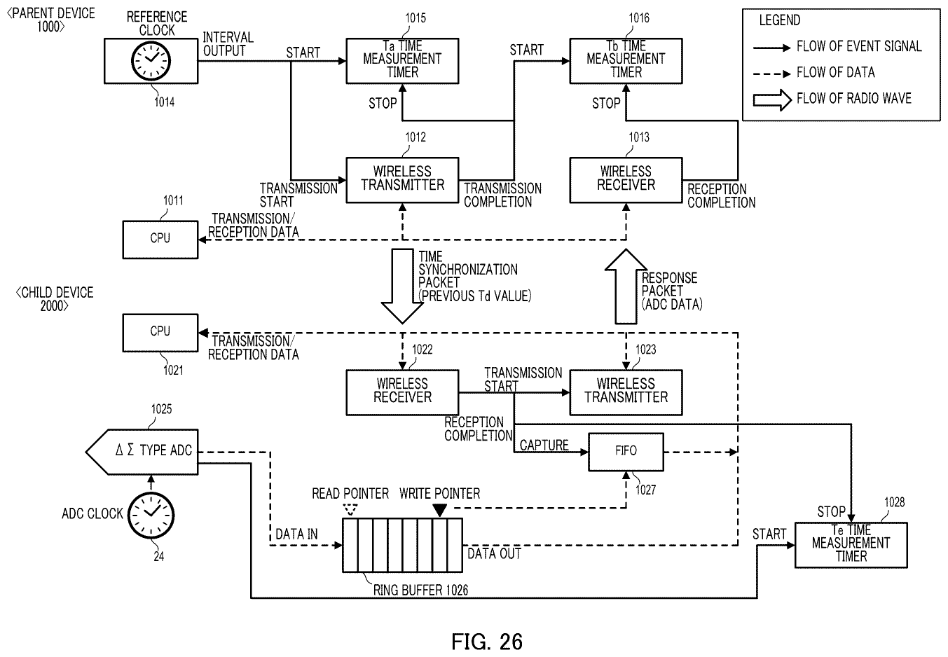

However, in Embodiment 3, in order to achieve higher accuracy time synchronization, the processing indicated below is proposed. FIG. 25 illustrates a processing example. It should be noted that 1 square in FIG. 25 represents 25 us. FIG. 26 is a block diagram illustrating a configuration example for performing the processing of FIG. 25. In the system illustrated in FIG. 26, client device 2000 includes Te time measurement timer 1028 as compared with the configuration described in FIG. 18. A peripheral built into a general-purpose microcontroller can be used as Te time measurement timer 1028, in the same manner as Ta time measurement timer 1015 and Tb time measurement timer 1016 of host device 1000, and therefore, there is no increase in the number of new components.

As can be seen from FIG. 25, Te time measurement timer 1028 measures time Te from the AD conversion completion interrupt to the radio wave reception. Client device 2000 calculates N by a new calculation expression using the time Te, N=ceil ((Td−Te)/125). Here, ceil means rounding off processing.

In this manner, in the case 1 of FIG. 25, since Te1=100, 1.8 obtained by (325−100)/125 is rounded off to 2. The position is shifted to “3” by “5”−2 and matches the timing of t1. In the case 2, since Te1=50, 2.2 obtained by (325−50)/125 is rounded off to 3. The position is shifted to “2” by “5”−3 and matches the timing of t1.

As described above, by introducing time Te, the deviation of less than one sample can be set to zero, and client device 2000 can wirelessly transmit the ADC data that is highly accurately synchronized with reference clock t1 of host device 1000.

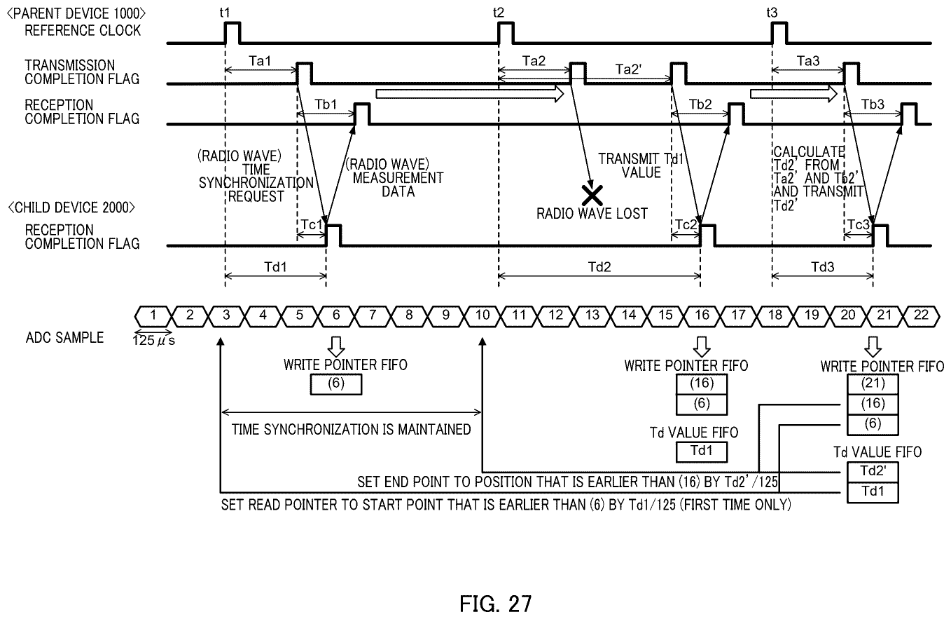

FIG. 27 is a processing example when a loss (packet loss) occurs in the radio waves transmitted from host device 1000 to client device 2000. Host device 1000 can recognize that the packet loss has occurred from the fact that there is no response from client device 2000. When host device 1000 recognizes that the packet loss has occurred, instead of retransmitting Td1 at the timing of next reference clock t3, host device 1000 transmits Td1 at a time after reference clock t2 by Ta2′. Client device 2000 puts the received value of Td1 into the Td value FIFO. In addition, since the ADC sample when Td1 is received is “16”, that is, the write pointer of ring buffer 26 is “16”, the value “16” is put into the write pointer FIFO.

When next reference clock t3 rises in host device 1000, host device 1000 performs the next transmission. At this time, host device 1000 calculates Td2′ based on Ta2′, Tb2, and Tc2 in the previous transmission/reception, sets the calculated value of Td2′ in the transmission data of wireless transmitter 1012, and transmits the value to client device 2000. Client device 2000 puts the received value of Td2′ into the Td value FIFO. In addition, since the ADC sample when Td2′ is received is “21”, that is, the write pointer of ring buffer 1026 is “21”, the value “21” is put into the write pointer FIFO.

When client device 2000 receives the Td value, the client device extracts the write pointer value “16” and Td2′ stored in FIFO 1027, sets the end point to a position that is earlier than the write pointer value “16” by N=Td2′/125 [pieces], and advances the read pointer while extracting the ADC data of ring buffer 1026 from the current position of the read pointer to the end point.

As described above, according to the time synchronization method of Embodiment 3, even when the packet loss occurs due to the radio wave deterioration, the time synchronization is maintained by retransmitting the Td value.

That is, when the response signal from the second wireless device (client device 2000) cannot be received, the first wireless device (host device 1000) transmits the retransmission signal to the second wireless device at time Ta′ (Ta2′ in the example of FIG. 27) later than time Ta. Further, the first wireless device acquires time Ta′ from the reference clock to the transmission of the retransmission signal to the second wireless device and time Tb (Tb2 in the example of FIG. 27) from the transmission of the retransmission signal to the reception of the response signal from the second wireless device, calculates time Td′ (Td2′ in the example of FIG. 27) from the reference clock to the reception of the retransmission signal by the second wireless device based on time Ta′ and time Tb, and transmits time Td′ to the second wireless device in the next transmission cycle of the retransmission signal, and the second wireless device transmits the data in a range corresponding to time Td′.

The synchronization method of Embodiment 3 may be applied to a case where there are one host device 1000 and a plurality of client devices 2000, and in this case as well, the synchronization error between one host device 1000 and the plurality of client devices 2000 can be suppressed.

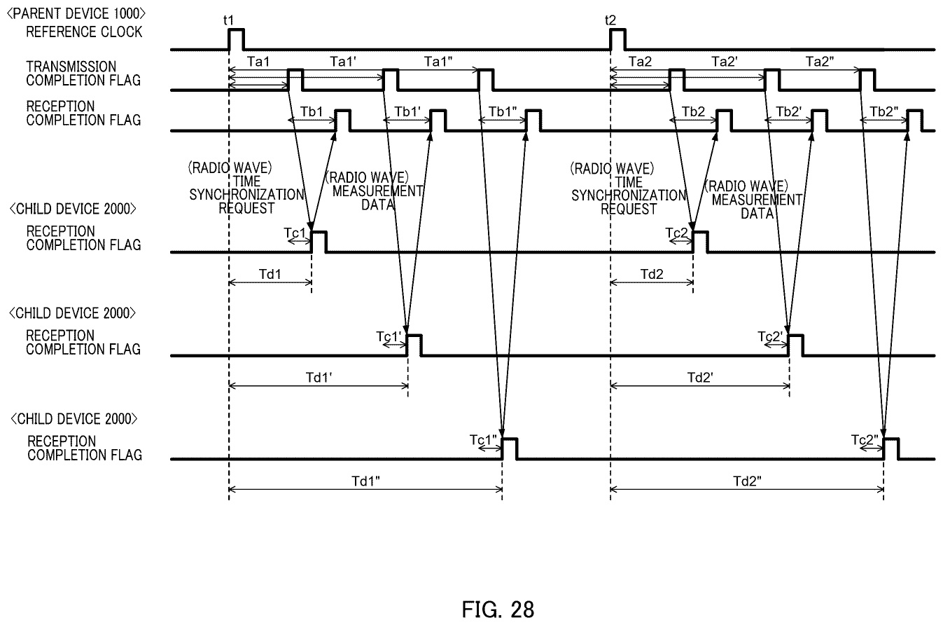

FIG. 28 illustrates an example in which the synchronization method of Embodiment 3 is applied to a system including a plurality of client devices. When there are a plurality of second wireless devices (client devices 2000a to 2000c), the first wireless device (host device 1000) acquires a plurality of times Ta (Ta, Ta′, and Ta″ in the example of FIG. 28) from the reference clock to the transmission of the first signal for the plurality of second wireless devices (client devices 2000a to 2000c) respectively, and a plurality of times Tb (Tb, Tb′, and Tb″ in the example of FIG. 28) from the transmission of the first signal to the reception of the response signals from the plurality of second wireless devices (client devices 2000a to 2000c) respectively.

Then, the first wireless device (host device 1000) calculates a plurality of times Td (Td, Td′, and Td″ in the example of FIG. 28) from the reference clock to the reception of the first signal by the plurality of second wireless devices (client devices 2000a to 2000c) respectively based on the plurality of times Ta (Ta, Ta′, and Ta″) and the plurality of times Tb (Tb, Tb′, and Tb″), and transmits the time Td corresponding to each second wireless device among the plurality of times Td (Td, Td′, and Td″) to the corresponding one of the plurality of second wireless devices (client devices 2000a to 2000c) in the next signal cycle of the first signal.

Each of the plurality of second wireless devices (client devices 2000a to 2000c) transmits the data in a range corresponding to the time Td (Td, Td′, or Td″) corresponding to the own machine, among the plurality of times Td (Td, Td′, and Td″).

As a result, host device 1000 can acquire the data in the range corresponding to the same interval of the reference clock from all client devices 2000a to 2000c. As a result, the synchronization error between one host device 1000 and the plurality of client devices 2000 can be suppressed.

### <3-2-2> Additional Processing

Here, as additional processing, a method of further increasing the accuracy of synchronizing the ADC data with the reference clock of host device 1000 by oversampling the ADC will be described.

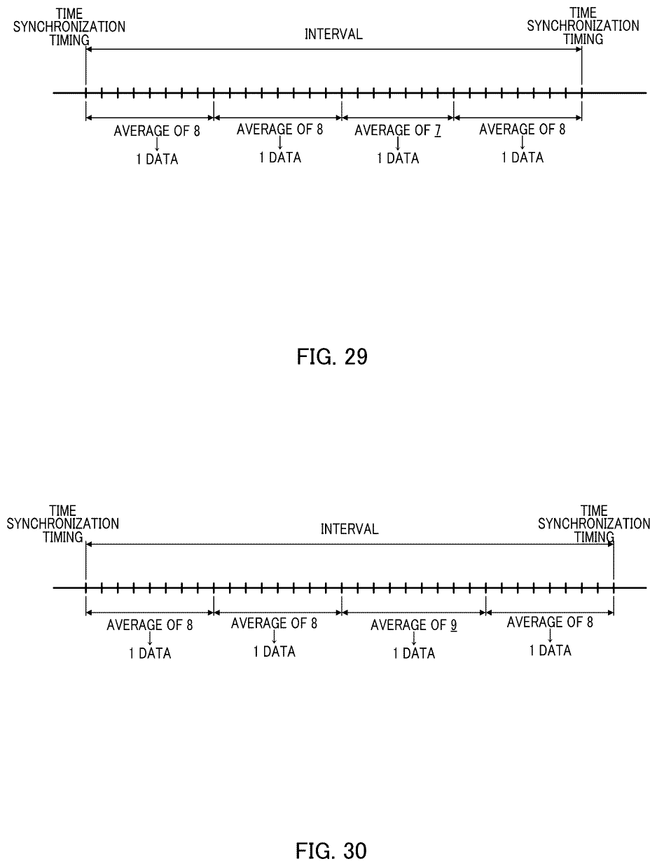

When a sampling rate required for sensor data is 1 kHz, the ADC is usually operated at 1 KHz. On the other hand, in Embodiment 3, ΔΣ type AD converter 1025 is operated in an oversampling manner at 8 kHz, which is 8 times the original sampling rate, and the data is downsampled as illustrated in FIG. 29. In addition, as is well known, the ΔΣ type AD converter has a configuration in which oversampling is easy in principle.

In this manner, the synchronization accuracy can be improved from 1 ms to 125 us.

Here, the interval of the time synchronization is generated by reference clock of the host device 1000, however, the ADC is operated by the clock (ADC clock) of client device 2000. The deviation occurs somewhere due to the clock accuracy of both sides.

FIG. 29 illustrates an example in which the ADC clock lags behind the reference clock, and is an example in which 32 samples are usually taken in one interval period, but occasionally 31 samples are taken. FIG. 30 illustrates an example in which the ADC clock advances faster than the reference clock, and is an example in which 32 samples are usually taken in one interval period, but occasionally 33 samples are taken.

By applying, for example, “Bresenham line drawing algorithm”, it is possible to automatically calculate a geometrically equal distribution of which data to average and how many pieces of data to average from two pieces of information: the ideal number of pieces of data when there was no clock drift in one interval period, and the actual number of pieces of measured data. In Embodiment 3, this processing is named “rubber band processing”.

The rubber band processing will be described in more detail.

When 32 samples are normally generated in one interval period, the 32 samples are downsampled to ⅛ to output a total of 4 data. On the other hand, as illustrated in FIG. 29, when 31 samples are generated in one interval period, 24 samples are downsampled to ⅛ to output 3 data, and 7 samples in between are downsampled to 1/7 to output 1 data, resulting in a total of 4 data being output. As illustrated in FIG. 30, when 33 samples are generated in one interval period, 24 samples are downsampled to ⅛ to output 3 data, and 9 samples in between are downsampled to 1/9 to output 1 data, resulting in a total of 4 data being output. As another example, for example, when 102 samples are generated in one interval period, 50 samples are downsampled to 1/25 to output 2 data, and 52 samples in between are downsampled to 1/26 to output 2 data, resulting in a total of 4 data being output.

The above-described examples are operation examples when the number of output data is set to 4. When the number of input samples is equal to or greater than the number of output data (in this case, four), the input samples are downsampled with an appropriate denominator and the number of output data (in this case, four) is obtained regardless of the number of input samples. When the number of input samples is not divisible by the number of output samples, the input samples may be divided geometrically evenly (linearly).

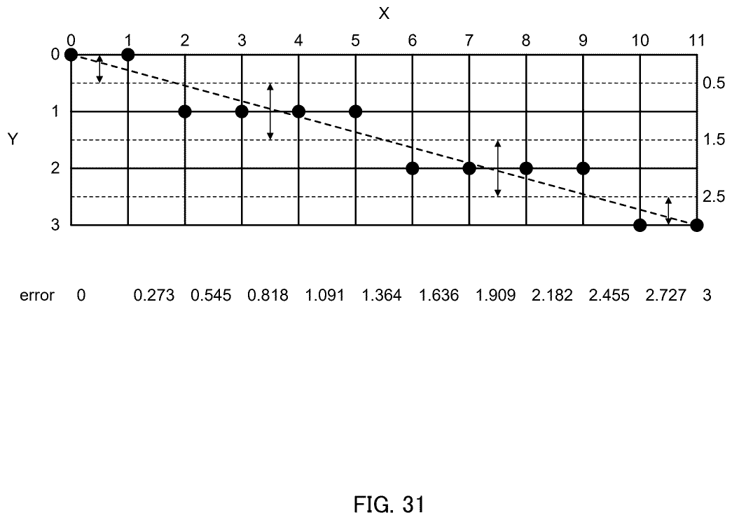

Meanwhile, in the algorithm of Bresenham used for the rubber band processing, as illustrated in FIG. 31, continuous points approximated to a straight line drawn from a given start point (0,0) to an end point (11,3) are obtained. However, since the number of input data is 12 and the number of output data is 4, the data should be divided into three equal parts, namely 12/4=3, however, the data is divided into two samples at both ends and four samples in the center portions, which is not uniform.

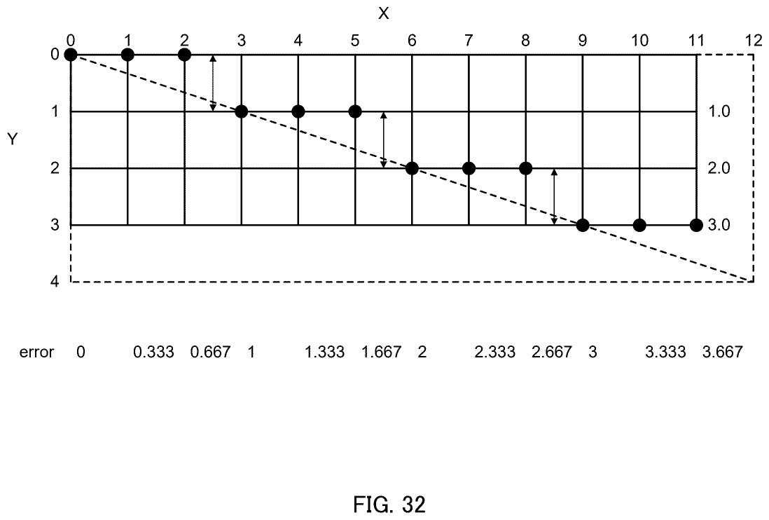

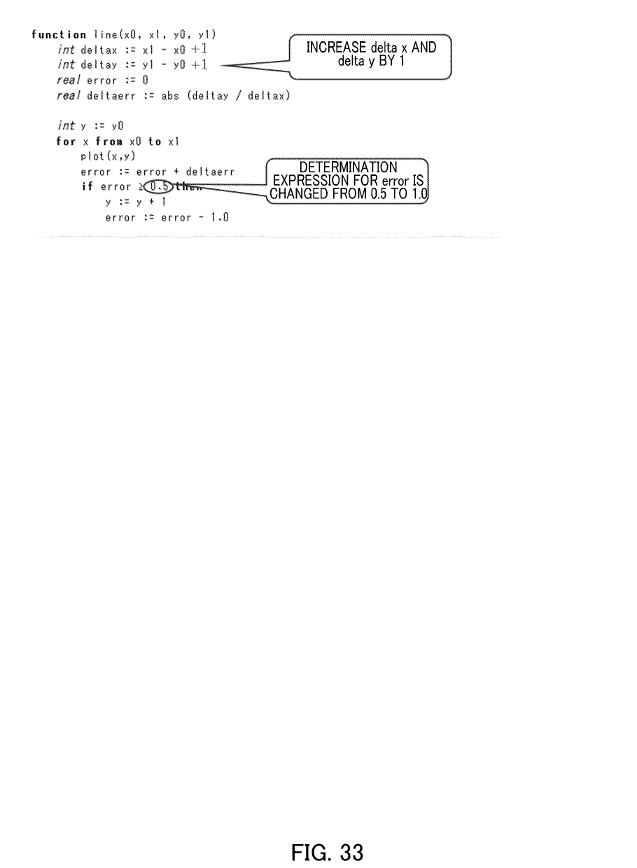

In Embodiment 3, the algorithm of Bresenham is modified. A modification example of the algorithm of Bresenham according to Embodiment 3 will be described with reference to FIG. 33. FIG. 33 illustrates an example of a program code (pseudo code) for implementing the Bresenham's algorithm. As illustrated in FIG. 33, each of the calculation expressions of delta x and delta y in the Bresenham's algorithm is increased by +1. In addition, the determination expression for the error is changed from 0.5 to 1.0. With this modification, as illustrated in FIG. 32, the 12 input data are divided into four equal parts of three samples each, and the four output data are obtained. As a matter of course, there are a plurality of variations in the program code of the Bresenham's algorithm in addition to the example of FIG. 33 due to optimization or simplification, and variables other than delta x, delta y, and error may be used. In any case, it is preferable to correct the Bresenham's algorithm in the same manner as described above.

Meanwhile, in a case of applying the time synchronization method of Embodiment 3 to a blood pressure pulse wave analyzing apparatus that calculates the pulse wave propagation velocity, the plurality of client devices 2000 correspond to the plurality of pulse wave sensors, and host device 1000 corresponds to the apparatus main body. When the time synchronization method of Embodiment 3 is applied to such a case, it is possible to synchronize low-quality data while maintaining real-time properties even in a case of a radio wave failure.

The description therefor will be made in detail. In a “measurement phase” in which the calculation of the pulse wave propagation velocity or blood pressure measurement is executed, it is necessary to transmit 1 ms of data without omission, however, in a “standby phase” in which a waveform is drawn on the screen, there is no significant impact even when several samples are missing. However, when the data for one interval period is simply missing, the baseline (straight line by interpolation) or a blank is displayed on the screen, and the shape of the original waveform is changed, which is not preferable as the display of the medical apparatus. However, when an attempt is made to retransmit the missing data, the data cannot be drawn on the screen until the retransmission is completed, and the real-time properties deteriorate.

Therefore, by using the above-described rubber band processing, the data for two intervals can be compressed (thinned out) into the data for one interval, so that the data for one interval, having information for two intervals, may be transmitted in the next interval without retransmission. In this manner, the real-time properties are maintained.

In the case of pulse wave, heart sound, and electrocardiogram waveform data, even when general compression such as ZIP is performed, the original data can only be compressed to about 80%, and the CPU processing load is also large. In the above-described rubber band processing, even when the number of pieces of input data is for two intervals or more, the number of pieces of data can always be compressed into the number of pieces of data for one interval, and the CPU processing load is also light and is fast because the processing is based on the above-described Bresenham's line drawing algorithm.

### <3-2-3> Application Example to Medical apparatus

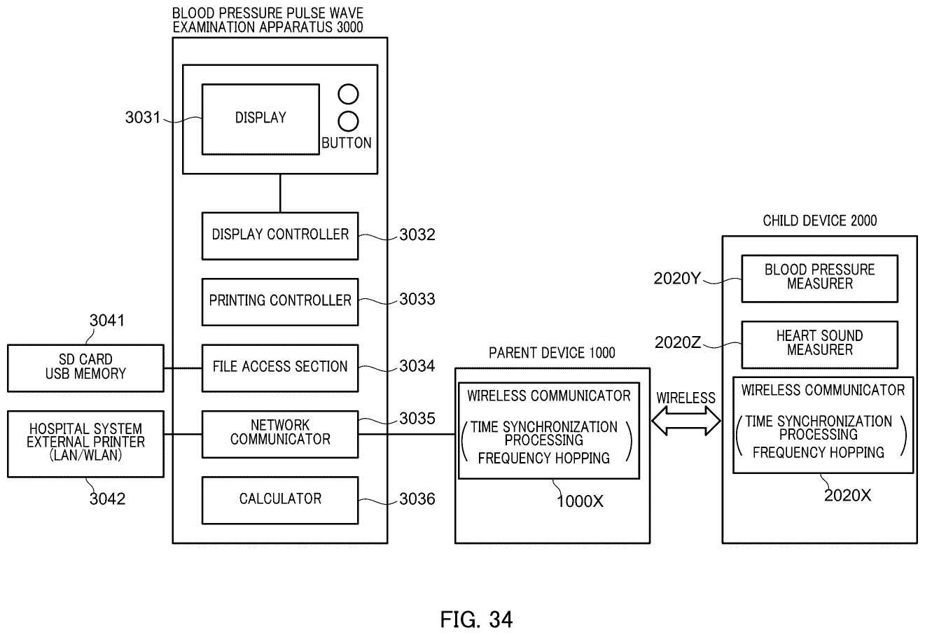

FIG. 34 illustrates an example in which the time synchronization method and the wireless system according to Embodiment 3 are applied to a medical apparatus. FIG. 34 illustrates an example in which host device 1000 and client device 2000 described above are used for the blood pressure pulse wave examination.

Blood pressure pulse wave analyzing apparatus 3000 includes display 3031, display controller 3032, printing controller 3033, file access section 3034, network controller 3035, and calculation unit 3036. External storage apparatus 3041, such as an SD card or a USB memory, is connected to file access section 3034. External system 3042, such as a hospital system or an external printer, is connected to the network communicator 3035 via a LAN or a WLAN.

Host device 1000 is connected in a wired manner to network communicator 3035. Host device 1000 includes wireless communicator 1000X that performs the time synchronization processing described with reference to FIG. 18. Wireless communicator 1000X has a frequency hopping function in addition to a time synchronization function.

Client device 2000 includes wireless communicator 2020X that performs the time synchronization processing described with reference to FIG. 18. Wireless communicator 2020X has a frequency hopping function in addition to a time synchronization function. Client device 2000 also includes blood pressure measurer 2020Y and heart sound measurer 2020Z. Client device 2000 transmits the blood pressure pulse wave measured by blood pressure measurer 2020Y and the heart sound measured by heart sound measurer 2020Z to host device 1000 via wireless communicator 2020X. Here, client device 2000 can also transmit the blood pressure pulse wave and the heart sound to host device 1000 simultaneously by transmitting the blood pressure pulse wave and the sound at different frequencies with the use of the frequency hopping function.

Host device 1000 receives the blood pressure pulse wave data and the heart sound data transmitted from client device 2000 and having a very small synchronization error by using the time synchronization method of Embodiment 3. Host device 1000 then transmits the received blood pressure pulse wave data and heart sound data to calculation unit 3036, so that calculation unit 3036 can obtain the high-accuracy pulse wave propagation velocity or the like.

### <3-3> Conclusion of Embodiment 3

As described above, Embodiment 3 provides a time synchronization method in a wireless system including the first wireless device (host device 1000) and the second wireless device (client device 2000) that performs wireless communication with the first wireless device (host device 1000), and the time synchronization method is for time-synchronizing transmission data of the second wireless device (client device 2000) with a reference clock of the first wireless device (host device 1000). In the time synchronization method, the first wireless device (host device 1000) measures time Ta from the reference clock to the transmission of a first signal to the second wireless device (client device 2000) and time Tb from the transmission of the first signal to the reception of a response signal from the second wireless device (client device 2000), calculates time Td from the reference clock to the reception of the first signal by the second wireless device (client device 2000) based on time Ta and time tb, and transmits time Td to the second wireless device (client device 2000) in the next transmission cycle of the first signal, and the second wireless device (client device 2000) transmits the data (measurement data) in the range corresponding to time Td.

As a result, it is possible to achieve the time synchronization method and the wireless system capable of suppressing the synchronization error between the wireless apparatuses (between host device 1000 and client device 2000) to, for example, 1 ms or less.

In Embodiment 3, high-resolution and high-accuracy ΔΣ type AD converter 1025 can be used in the untethered clock (asynchronous).

In addition, according to Embodiment 3, by using the rubber band, the clock drift between host device 1000 and client device 2000 can be reduced.

In addition, according to Embodiment 3, accurate resynchronization can be performed even in a case of a radio wave failure.

In addition, according to Embodiment 3, by using the rubber band, it is possible to synchronize low-quality data while maintaining the real-time properties even in a case of a radio wave failure.

Further, according to Embodiment 3, a dedicated apparatus is not required during implementation, and peripherals built into a general-purpose one-chip microcontroller, such as an interval clock, a timer, a register, and a ring buffer, can be used, so that the implementation is easy.

In particular, according to Embodiment 3, it is not necessary to use a high-accuracy oscillator for the ADC clock. The reason for this will be described. The deviation between the clock on the host device side and the ADC clock of the client device is smaller as the frequency accuracy is higher. In general, there are a temperature-compensated quartz oscillator (TCXO), a quartz oscillator with a temperature-controlled chamber (OCXO), and the like as a high-accuracy clock oscillation source, however, these are specialized components and are expensive. In the synchronization method of Embodiment 3, since the rubber band has a function of reducing the clock drift, a general-purpose quartz oscillator can be used as the clock oscillation source instead of the high-accuracy oscillator.

Embodiment 3 described above is merely an example of concretization in implementing the present invention, and the technical scope of the present invention should not be construed as being limited by these. That is, the present invention can be carried out in various forms without departing from the spirit and the main features thereof.

Embodiment 3 describes the case in which ring buffer 1026 is provided as the memory that stores the data after the AD conversion, however, the present disclosure is not limited thereto, and a memory other than ring buffer 1026 may be provided. However, ring buffer 1026 has an advantage that it requires only a small memory capacity.

Embodiment 3 describes the case in which the position of the write pointer of ring buffer 1026 and time Td are stored in FIFO 1027, however, the present disclosure is not limited thereto, and the function of FIFO 1027 may be implemented by a memory and a program.

That is, one aspect of the present disclosure has the following features. The second wireless device (client device 2000) includes an AD converter (in the example of Embodiment 3, ΔΣ type AD converter 1025) that performs the AD conversion on the transmission information, the first memory (in the example of Embodiment 3, ring buffer 1026) that stores the data after the AD conversion, and the second memory (in the example of Embodiment 3, FIFO memory 1027). The second wireless device stores, in the second memory, the position of the write pointer of the first memory when the first signal is received and time Td indicated by the received first signal, and the second wireless device controls the range of the data to be transmitted by setting the readout position from the first memory and forming the transmission data based on the position of the write pointer and time Td stored in the second memory.

Embodiment 3 describes the case in which the first wireless device (host device 1000) measures time Ta that is a time from the reference clock to the transmission of the first signal to the second wireless device (client device 2000) and time Tb that is a time from the transmission of the first signal to the reception of the response signal from the second wireless device, however, since time Tb is a very small value, time Tb may be used as a fixed value without being directly measured depending on an allowable error of the wireless system in which the synchronization method is adopted.
(1) One aspect of a synchronization method according to the present disclosure is a synchronization method for synchronizing transmission data of a second wireless device with a reference clock of a first wireless device, the synchronization method including:
acquiring, by the first wireless device, a time Ta that is a time from the reference clock to transmission of a first signal to the second wireless device and a time Tb that is a time from the transmission of the first signal to reception of a response signal from the second wireless device;
calculating, by the first wireless device, a time Td that is a time from the reference clock to reception of the first signal by the second wireless device based on the time Ta and the time Tb;
transmitting, by the first wireless device, the time Td to the second wireless device in a next transmission cycle of the first signal; and
transmitting, by the second wireless device, data in a range corresponding to the time Td.
(2) One aspect of the synchronization method according to the present disclosure is as follows in the aspect (1):
the second wireless device calculates timing of the reference clock of the first wireless device from the time Td and transmits data in a range corresponding to an interval of the reference clock.
(3) One aspect of the synchronization method according to the present disclosure is as follows in the aspect (1):
the second wireless device includes an AD converter that performs AD conversion on transmission information, a first memory that stores data after the AD conversion, and a second memory;
the second memory stores a position of a write pointer of the first memory when the first signal is received and the time Td indicated by the received first signal; and
the second wireless device controls a range of data to be transmitted by setting a readout position from the first memory, based on the stored position of the write pointer and the stored time Td.
(4) One aspect of the synchronization method according to the present disclosure is as follows in the aspect (1):
the second wireless device sets, as an end point of the readout position, AD conversion data that is earlier than the position of the write pointer stored in the second memory by a result of dividing the time Td by a sampling period of the AD converter.
(5) One aspect of the synchronization method according to the present disclosure is as follows in the aspect (3) or (4):
the AD converter is a ΔΣ type AD converter.
(6) One aspect of the synchronization method according to the present disclosure is as follows in the aspect (5):
the ΔΣ type AD converter oversamples the transmission information, and
the second wireless device downsamples data after the oversampling.
(7) One aspect of the synchronization method according to the present disclosure is as follows in the aspect (1):
when the first wireless device cannot receive the response signal from the second wireless device, the first wireless device transmits a retransmission signal to the second wireless device at a time Ta′ later than the time Ta;
the first wireless device further acquires the time Ta′ and a time Tb, the time Ta′ being a time from the reference clock to transmission of the retransmission signal to the second wireless device, the time Tb being a time from the transmission of the retransmission signal to the reception of the response signal from the second wireless device;
the first wireless device calculates, based on the time Ta′ and the time Tb, a time Td′ that is a time from the reference clock to the reception of the retransmission signal by the second wireless device;
the first wireless device transmits the time Td′ to the second wireless device in the next transmission cycle of the retransmission signal; and
the second wireless device transmits data in a range corresponding to the time Td′.
(8) One aspect of the synchronization method according to the present disclosure is as follows in the aspect (1):
when a plurality of the second wireless devices are present,
   the first wireless device acquires a plurality of times Ta that are times from the reference clock to the transmission of the first signal to the plurality of second wireless devices, respectively, and a plurality of times Tb that are times from the transmission of the first signal to the reception of the response signal from the plurality of second wireless devices, respectively,
   the first wireless device calculates, based on the plurality of times Ta and the plurality of times Tb, a plurality of times Td that are times from the reference clock to the reception of the first signal by the plurality of second wireless devices, respectively,
   the first wireless device transmits a time Td corresponding to each of the plurality of second wireless devices to the corresponding one of the plurality of second wireless devices in the next transmission cycle of the first signal, the time Td being among the plurality of times Td; and
   each of the plurality of second wireless devices transmits data in a range corresponding to the time Td corresponding to the own second wireless device, the time Td being among the plurality of times Td.
(9) One aspect of the synchronization method according to the present disclosure is as follows in the aspect (3):
causing the first memory to be a ring buffer.
(10) One aspect of the synchronization method according to the present disclosure is as follows in the aspect (3):
the second memory is a FIFO memory.
(11) One aspect of the synchronization method according to the present disclosure is as follows in the aspect (1):
the first wireless device is a host device in a medical apparatus,
the second wireless device is provided in a biological information sensor of the medical apparatus, and
the data transmitted by the second wireless device is biological data obtained by the biological information sensor.
(12) One aspect of a wireless system according to the present disclosure is a wireless system including:
a first wireless device; and
a second wireless device that performs wireless communication with the first wireless device, in which
the first wireless device acquires a time Ta that is a time from the reference clock to transmission of a first signal to the second wireless device and a time Tb that is a time from the transmission of the first signal to reception of a response signal from the second wireless device,
the first wireless device calculates a time Td that is a time from the reference clock to reception of the first signal by the second wireless device based on the time Ta and the time Tb,
the first wireless device transmits the time Td to the second wireless device in a next transmission cycle of the first signal, and

the second wireless device transmits data in a range corresponding to the time Td.
(13) One aspect of the wireless system according to the present disclosure is as follows in the aspect (12):
the second wireless device calculates timing of the reference clock of the first wireless device from the time Td; and
the second wireless device transmits data in a range corresponding to an interval of the reference clock.
(14) One aspect of the wireless system according to the present disclosure is as follows in the aspect (12):
the second wireless device includes an AD converter that performs AD conversion on transmission information, a first memory that stores data after the AD conversion, and a second memory;
the second memory stores a position of a write pointer of the first memory when the first signal is received and the time Td indicated by the received first signal; and
the second wireless device controls, based on the stored position of the write pointer and the stored time Td, a range of data to be transmitted by setting a readout position from the first memory.
(15) One aspect of the wireless system according to the present disclosure is as follows in the aspect (14):
the second wireless device sets, as an end point of the readout position, AD conversion data that is earlier than the position of the write pointer stored in the second memory by a result of dividing the time Td by a sampling period of the AD converter.
(16) One aspect of a first wireless device according to the present disclosure is a first wireless device that performs synchronous communication with a second wireless device, the first wireless device including:
a timer that acquires a time Ta that is a time from a reference clock to transmission of a first signal to the second wireless device and a time Tb that is a time from the transmission of the first signal to reception of a response signal from the second wireless device,
a calculation unit that calculates a time Td that is a time from the reference clock to reception of the first signal by the second wireless device based on the time Ta and the time Tb, and
a wireless transmitter that transmits the time Td to the second wireless device in a next transmission cycle of the first signal.
(17) One aspect of a second wireless device according to the present disclosure is a second wireless device that performs synchronous communication with a first wireless device (in which the first wireless device acquires a time Ta that is a time from a reference clock to transmission of a first signal to the second wireless device and a time Tb that is a time from the transmission of the first signal to reception of a response signal from the second wireless device, and the first wireless device calculates a time Td that is a time from the reference clock to reception of the first signal by the second wireless device based on the time Ta and the time Tb, and the first wireless device transmits the time Td to the second wireless device in a next transmission cycle of the first signal), the second wireless device including:
an AD converter that performs AD conversion on transmission information,
a first memory that stores data after the AD conversion,
a second memory that stores a position of a write pointer of the first memory when the first signal is received from the first wireless device and the time Td indicated by the received first signal, and
a wireless transmitter that controls a range of data to be transmitted by setting a readout position from the first memory based on the stored position of the write pointer and the stored time Td.

### <4> Conclusion

The techniques described in each of the above-described embodiments can also be implemented in combination with each other. The blood pressure pulse wave analyzing system according to the present disclosure can have the following aspects.
(1) One aspect of a blood pressure pulse wave analyzing system according to the present disclosure includes the following:
a plurality of wireless cuff devices each to be mounted on a predetermined site of a subject; and
a blood pressure pulse wave analyzing apparatus that performs wireless communication with the plurality of wireless cuff devices, controls the plurality of wireless cuff devices, and collects pulse wave data from the plurality of wireless cuff devices to examine a blood vessel state of the subject, in which
each of the plurality of wireless cuff devices includes
   a cuff to be wound around the predetermined site of the subject, and
   a wireless communication unit attached to the cuff.
(2) As described in Embodiment 1, one aspect of a blood pressure pulse wave analyzing system according to the present disclosure is as follows in the aspect (1):
the blood pressure pulse wave analyzing apparatus includes
   an arrival direction estimator that estimates an arrival direction of a radio wave from each of the plurality of wireless cuff devices,
   a distance estimator that estimates a distance to each of the plurality of wireless cuff devices, and
   a mounting site estimator that estimates a mounting site of each of the plurality of wireless cuff devices on the subject based on the estimated arrival direction and the estimated distance.
(3) As described in Embodiment 1, one aspect of a blood pressure pulse wave analyzing system according to the present disclosure is as follows in the aspect (2):
the blood pressure pulse wave analyzing apparatus further includes
   a determiner that determines validity of the mounting site of each of the plurality of wireless cuff devices based on the estimated mounting site, and
   an output that outputs a determination result of the determiner.
(4) As described in Embodiment 1, one aspect of a blood pressure pulse wave analyzing system according to the present disclosure is as follows in the aspect (2) or (3):
the blood pressure pulse wave analyzing apparatus and the plurality of wireless cuff devices each include a UWB communicator; and
the arrival direction estimator and the distance estimator of the blood pressure pulse wave analyzing apparatus estimate the arrival direction and the distance based on a radio wave in a UWB communication method.
(5) As described in Embodiment 2, one aspect of a blood pressure pulse wave analyzing system according to the present disclosure is as follows in the aspect (1):
the cuff includes an NFC tag having mounting site information; and the wireless communication unit is detachably attached to the cuff, is wirelessly connected to the NFC tag, and wirelessly transmits, to the blood pressure pulse wave analyzing apparatus, the mounting site information and data obtained by driving the cuff.
(6) As described in Embodiment 2, one aspect of a blood pressure pulse wave analyzing system according to the present disclosure is as follows in the aspect (s):
prior to transmission of the data, the wireless communication unit wirelessly transmits information on the cuff to the blood pressure pulse wave analyzing apparatus, the information including the mounting site information.
(7) As described in Embodiment 2, one aspect of a blood pressure pulse wave analyzing system according to the present disclosure is as follows in the aspect (6):
in addition to the mounting site information, a packet wirelessly transmitted to the blood pressure pulse wave analyzing apparatus by the wireless communication unit prior to the transmission of the data includes at least one of size information of the cuff and type information of the cuff.
(8) As described in Embodiment 2, one aspect of a blood pressure pulse wave analyzing system according to the present disclosure is as follows in the aspect (5):
the cuff includes an air bag and is to be wound around the predetermined site of the subject; and
the wireless communication unit further includes a cuff driver that supplies and discharges air to and from the air bag of the cuff, and a cuff pressure detector that detects a cuff pressure of the cuff during an examination.
(9) As described in Embodiment 2, one aspect of a blood pressure pulse wave analyzing system according to the present disclosure is as follows in the aspect (8):
the cuff driver and the cuff pressure detector are provided in the wireless communication unit.
(10) As described in Embodiment 2, one aspect of a blood pressure pulse wave analyzing system according to the present disclosure is as follows in the aspect (8):
the wireless communication unit at least wirelessly receives, from the blood pressure pulse wave analyzing apparatus, a control signal for controlling the cuff driver and wirelessly transmits, to the blood pressure pulse wave analyzing apparatus, the mounting site information and data on the cuff pressure obtained by driving the cuff.
(11) As described in Embodiment 2, one aspect of a blood pressure pulse wave analyzing system according to the present disclosure is as follows in the aspect (5):
the NFC tag includes a storage, and the storage stores size information of the cuff and/or type information of the cuff in addition to the mounting site information.
(12) As described in Embodiment 2, one aspect of a blood pressure pulse wave analyzing system according to the present disclosure is as follows in the aspect (8):
the NFC tag includes a storage, and the storage stores a limit pressurization number of the cuff; and
the wireless communication unit writes, into the storage, an actual pressurization number that is the number of times a pressure in the cuff is increased by the cuff driver, and when the actual pressurization number becomes equal to or greater than the limit pressurization number, the wireless communication unit outputs an alarm indicating that the actual pressurization number becomes equal to or greater than the limit pressurization number.
(13) As described in Embodiment 1, one aspect of a blood pressure pulse wave analyzing system according to the present disclosure is as follows in the aspect (1):
the blood pressure pulse wave analyzing apparatus includes
   a distance estimator that estimates a distance to each of the plurality of wireless cuff devices, and
   a selector that selects a wireless cuff device located within a predetermined distance range based on the estimated distance, the wireless cuff device being among the plurality of wireless cuff devices.
(14) As described in Embodiment 1, one aspect of a blood pressure pulse wave analyzing system according to the present disclosure is as follows in the aspect (13):
the blood pressure pulse wave analyzing apparatus excludes, from a pairing target, a wireless cuff device other than the wireless cuff device selected by the selector even when a wireless signal from the excluded wireless cuff device is received.
(15) As described in Embodiment 1, one aspect of a blood pressure pulse wave analyzing system according to the present disclosure is as follows in the aspect (13):
the blood pressure pulse wave analyzing apparatus includes
   an arrival direction estimator that estimates an arrival direction of a radio wave from each of the plurality of wireless cuff devices,
   a distance estimator that estimates a distance to each of the plurality of wireless cuff devices,
   a blood vessel length estimator that estimates a blood vessel length between the plurality of wireless cuff devices on the subject based on the estimated arrival direction and the estimated distance regarding the plurality of wireless cuff devices, and
   a calculation unit that calculates an indicator indicating the blood vessel state of the subject based on the estimated blood vessel length and the pulse wave data from the plurality of wireless cuff devices.
(16) As described in Embodiment 1, one aspect of a blood pressure pulse wave analyzing system according to the present disclosure is as follows in the aspect (15):
a wireless sensor that is mounted on the subject at a position corresponding to a heart of the subject, detects a behavior of the heart, and wirelessly transmits heart behavior information, the behavior being a source of a pulse wave, in which
the arrival direction estimator estimates the arrival direction of the radio wave from each of the plurality of wireless cuff devices and an arrival direction of a radio wave from the wireless sensor,
the distance estimator estimates the distance to each of the plurality of wireless cuff devices and a distance to the wireless sensor,
the blood vessel length estimator estimates a blood vessel length of the subject between each of the plurality of wireless cuff devices and the wireless sensor based on the estimated arrival directions and the estimated distances regarding the plurality of wireless cuff devices and the wireless sensor, and
the calculation unit calculates the indicator indicating the blood vessel state of the subject based on the estimated blood vessel length, the pulse wave data, and the heart behavior information.
(17) As described in Embodiment 1, one aspect of a blood pressure pulse wave analyzing system according to the present disclosure is as follows in the aspect (15):
the plurality of wireless cuff devices obtain a distance between the plurality of wireless cuff devices wirelessly, and wirelessly transmit information on the distance to the blood pressure pulse wave analyzing apparatus.
(18) As described in Embodiment 1, one aspect of a blood pressure pulse wave analyzing system according to the present disclosure is as follows in the aspect (15):
a wireless sensor that is mounted on the subject at a position corresponding to a heart of the subject, detects a behavior of the heart, and wirelessly transmits heart behavior information, the behavior being a source of a pulse wave, in which
the wireless sensor and each of the plurality of wireless cuff devices obtain a distance between the wireless sensor and each of the plurality of wireless cuff devices wirelessly, and wirelessly transmit information on the distance to the blood pressure pulse wave analyzing apparatus.
(19) As described in Embodiment 3, one aspect of a blood pressure pulse wave analyzing system according to the present disclosure is as follows in the aspect (1):
the blood pressure pulse wave analyzing apparatus is set as a first wireless device, and a wireless cuff device of the plurality of wireless cuff devices is set as a second wireless device, and transmission data of the second wireless device is synchronized with a reference clock of the first wireless device and transmitted;
the first wireless device acquires a time Ta that is a time from the reference clock to transmission of a first signal to the second wireless device and a time Tb that is a time from the transmission of the first signal to reception of a response signal from the second wireless device;
the first wireless device calculates a time Td that is a time from the reference clock to reception of the first signal by the second wireless device based on the time Ta and the time Tb;
the first wireless device transmits the time Td to the second wireless device in a next transmission cycle of the first signal and
the second wireless device transmits data in a range corresponding to the time Td.
(20) As described in Embodiment 3, one aspect of a blood pressure pulse wave analyzing system according to the present disclosure is as follows in the aspect (19):
the second wireless device calculates timing of the reference clock of the first wireless device from the time Td; and
the second wireless device transmits data in a range corresponding to an interval of the reference clock.
(21) As described in Embodiment 3, one aspect of a blood pressure pulse wave analyzing system according to the present disclosure is as follows in the aspect (19):
the second wireless device includes an AD converter, a first memory, and a second memory, the AD converter performing AD conversion on transmission information, the first memory storing data after the AD conversion;
the second memory stores a position of a write pointer of the first memory when the first signal is received and the time Td indicated by the received first signal; and
the second wireless device controls, based on the stored position of the write pointer and the stored time Td, a range of data to be transmitted by setting a readout position from the first memory.
(22) As described in Embodiment 3, one aspect of a blood pressure pulse wave analyzing system according to the present disclosure is as follows in the aspect (21):
the second wireless device sets, as an end point of the readout position, AD conversion data that is earlier than the position of the write pointer stored in the second memory by a result of dividing the time Td by a sampling period of the AD converter.
(23) As described in Embodiment 3, one aspect of a blood pressure pulse wave analyzing system according to the present disclosure is as follows in the aspect (21) or (22): the AD converter is a ΔΣ type AD converter.
(24) As described in Embodiment 3, one aspect of a blood pressure pulse wave analyzing system according to the present disclosure is as follows in the aspect (23):
the ΔΣ type AD converter oversamples the transmission information; and
the second wireless device downsamples data after the oversampling.
(25) As described in Embodiment 3, one aspect of a blood pressure pulse wave analyzing system according to the present disclosure is as follows in the aspect (19):
when the first wireless device fails to receive the response signal from the second wireless device, the first wireless device transmits a retransmission signal to the second wireless device at a time Ta′ later than the time Ta;
the first wireless device further acquires the time Ta′ and a time Tb, the time Ta′ being a time from the reference clock to transmission of the retransmission signal to the second wireless device, the time Tb being a time from the transmission of the retransmission signal to the reception of the response signal from the second wireless device;
the first wireless device calculates, based on the time Ta′ and the time Tb, a time Td′ that is a time from the reference clock to the reception of the retransmission signal by the second wireless device;
the first wireless device transmits the time Td′ to the second wireless device in the next transmission cycle of the retransmission signal; and
the second wireless device transmits data in a range corresponding to the time Td′.
(26) As described in Embodiment 3, one aspect of a blood pressure pulse wave analyzing system according to the present disclosure is as follows in the aspect (19):
when the plurality of wireless cuff devices are set as a plurality of second wireless devices,
   the first wireless device acquires a plurality of times Ta that are times from the reference clock to the transmission of the first signal to the plurality of second wireless devices, respectively, and a plurality of times Tb that are times from the transmission of the first signal to the reception of the response signal from the plurality of second wireless devices, respectively,
   the first wireless device calculates, based on the plurality of times Ta and the plurality of times Tb, a plurality of times Td that are times from the reference clock to the reception of the first signal by the plurality of second wireless devices, respectively,
   the first wireless device transmits a time Td corresponding to each of the plurality of second wireless devices to a corresponding one of the plurality of second wireless devices in the next transmission cycle of the first signal, the time Td being among the plurality of times Td, and
   each of the plurality of second wireless devices transmits data in a range corresponding to the time Td corresponding to the own second wireless device, the time Td being among the plurality of times Td.
(27) As described in Embodiment 3, one aspect of a blood pressure pulse wave analyzing system according to the present disclosure is as follows in the aspect (21): the first memory is a ring buffer.
(28) As described in Embodiment 3, one aspect of a blood pressure pulse wave analyzing system according to the present disclosure is as follows in the aspect (21): the second memory is a FIFO memory.

The disclosure contents of the specification, the drawings, and the abstract of each of Japanese Patent Applications No. 2023-58496, No. 2023-58512, No. 2023-58517, No. 2023-58525, and No. 2023-58529 filed on Mar. 31, 2023 are incorporated herein by reference in their entireties.

### INDUSTRIAL APPLICABILITY

The technique of the present disclosure is suitable for a blood pressure pulse wave analyzing system or the like.

### REFERENCE SIGNS LIST

10 (10-1 to 10-4), 10′ (10-1′ to 10-4′) Wireless cuff device
20 (20-1 to 20-4), 20′ (20-1′ to 20-4′) Cuff
20a Edge
30 (30-1 to 30-4) Wireless tag
100 Wireless communication unit
110 Battery
120 Wireless power feeder
130 User interface
140 Cuff driver
150 Cuff pressure detector
160, 160′ Wireless communicator
161 Memory
162 UWB communication module
170, 1011, 1021 CPU
200, 200a, 200b Blood pressure pulse wave analyzing apparatus
210 UWB communication module
220 Calculation controller
221 Controller
222 Calculation unit
223 Arrival direction estimator
224 Distance estimator
225 Mounting site estimator
226 Determiner
230 User interface
301 Blood vessel length estimator
400 Wireless heart sound sensor
1012, 1023 Wireless transmitter
1013, 1022 Wireless receiver
1014 Reference clock generator
1015 Ta time measurement timer
1016 Tb time measurement timer
1024 ADC clock generator
1025 ΔΣ type AD converter
1026 Ring buffer
1027 FIFO memory
1028 Te time measurement timer
2161 NFC communication module
2162 Bluetooth communication module

What is claim is:

1.-28. (canceled)

29. A blood pressure pulse wave analyzing system, comprising:
a plurality of wireless cuff devices each to be mounted on a predetermined site of a subject; and
a blood pressure pulse wave analyzing apparatus that performs wireless communication with the plurality of wireless cuff devices, controls the plurality of wireless cuff devices, and collects pulse wave data from the plurality of wireless cuff devices to examine a blood vessel state of the subject, wherein
each of the plurality of wireless cuff devices includes
a cuff to be wound around the predetermined site of the subject, and
a wireless communication unit attached to the cuff.

30. The blood pressure pulse wave analyzing system according to claim 29, wherein
the blood pressure pulse wave analyzing apparatus includes
an arrival direction estimator that estimates an arrival direction of a radio wave from each of the plurality of wireless cuff devices,
a distance estimator that estimates a distance to each of the plurality of wireless cuff devices, and
a mounting site estimator that estimates a mounting site of each of the plurality of wireless cuff devices on the subject based on the estimated arrival direction and the estimated distance.

31. The blood pressure pulse wave analyzing system according to claim 30, wherein
the blood pressure pulse wave analyzing apparatus further includes
a determiner that determines validity of the mounting site of each of the plurality of wireless cuff devices based on the estimated mounting site, and
an output that outputs a determination result of the determiner.

32. The blood pressure pulse wave analyzing system according to claim 30, wherein:
the blood pressure pulse wave analyzing apparatus and the plurality of wireless cuff devices each include a UWB communicator; and
the arrival direction estimator and the distance estimator of the blood pressure pulse wave analyzing apparatus estimate the arrival direction and the distance based on a radio wave in a UWB communication method.

33. The blood pressure pulse wave analyzing system according to claim 30, wherein:
the cuff includes an NFC tag having mounting site information; and
the wireless communication unit is detachably attached to the cuff, is wirelessly connected to the NFC tag, and wirelessly transmits, to the blood pressure pulse wave analyzing apparatus, the mounting site information and data obtained by driving the cuff.

34. The blood pressure pulse wave analyzing system according to claim 33, wherein
prior to transmission of the data, the wireless communication unit wirelessly transmits information on the cuff to the blood pressure pulse wave analyzing apparatus, the information including the mounting site information.

35. The blood pressure pulse wave analyzing system according to claim 34, wherein
in addition to the mounting site information, a packet wirelessly transmitted to the blood pressure pulse wave analyzing apparatus by the wireless communication unit prior to the transmission of the data includes at least one of size information of the cuff and type information of the cuff.

36. The blood pressure pulse wave analyzing system according to claim 33, wherein:
the cuff includes an air bag and is to be wound around the predetermined site of the subject; and
the wireless communication unit further includes a cuff driver that supplies and discharges air to and from the air bag of the cuff, and a cuff pressure detector that detects a cuff pressure of the cuff during an examination.

37. The blood pressure pulse wave analyzing system according to claim 36, wherein
the cuff driver and the cuff pressure detector are provided in the wireless communication unit.

38. The blood pressure pulse wave analyzing system according to claim 36, wherein
the wireless communication unit at least wirelessly receives, from the blood pressure pulse wave analyzing apparatus, a control signal for controlling the cuff driver and wirelessly transmits, to the blood pressure pulse wave analyzing apparatus, the mounting site information and data on the cuff pressure obtained by driving the cuff.

39. The blood pressure pulse wave analyzing system according to claim 33, wherein
the NFC tag includes a storage, and the storage stores size information of the cuff and/or type information of the cuff in addition to the mounting site information.

40. The blood pressure pulse wave analyzing system according to claim 36, wherein:
the NFC tag includes a storage, and the storage stores a limit pressurization number of the cuff; and
the wireless communication unit writes, into the storage, an actual pressurization number that is the number of times a pressure in the cuff is increased by the cuff driver, and when the actual pressurization number becomes equal to or greater than the limit pressurization number, the wireless communication unit outputs an alarm indicating that the actual pressurization number becomes equal to or greater than the limit pressurization number.

41. The blood pressure pulse wave analyzing system according to claim 30, wherein
the blood pressure pulse wave analyzing apparatus further includes a selector that selects a wireless cuff device located within a predetermined distance range based on the estimated distance, the wireless cuff device being among the plurality of wireless cuff devices.

42. The blood pressure pulse wave analyzing system according to claim 41, wherein
the blood pressure pulse wave analyzing apparatus excludes, from a pairing target, a wireless cuff device other than the wireless cuff device selected by the selector even when a wireless signal from the excluded wireless cuff device is received.

43. The blood pressure pulse wave analyzing system according to claim 29, wherein
the blood pressure pulse wave analyzing apparatus includes
an arrival direction estimator that estimates an arrival direction of a radio wave from each of the plurality of wireless cuff devices,
a distance estimator that estimates a distance to each of the plurality of wireless cuff devices,
a blood vessel length estimator that estimates a blood vessel length between the plurality of wireless cuff devices on the subject based on the estimated arrival direction and the estimated distance regarding the plurality of wireless cuff devices, and
a calculation unit that calculates an indicator indicating the blood vessel state of the subject based on the estimated blood vessel length and the pulse wave data from the plurality of wireless cuff devices.

44. The blood pressure pulse wave analyzing system according to claim 43, further comprising:
a wireless sensor that is mounted on the subject at a position corresponding to a heart of the subject, detects a behavior of the heart, and wirelessly transmits heart behavior information, the behavior being a source of a pulse wave, wherein
the arrival direction estimator estimates the arrival direction of the radio wave from each of the plurality of wireless cuff devices and an arrival direction of a radio wave from the wireless sensor,
the distance estimator estimates the distance to each of the plurality of wireless cuff devices and a distance to the wireless sensor,
the blood vessel length estimator estimates a blood vessel length of the subject between each of the plurality of wireless cuff devices and the wireless sensor based on the estimated arrival directions and the estimated distances regarding the plurality of wireless cuff devices and the wireless sensor, and
the calculation unit calculates the indicator indicating the blood vessel state of the subject based on the estimated blood vessel length, the pulse wave data, and the heart behavior information.

45. The blood pressure pulse wave analyzing system according to claim 43, wherein
the plurality of wireless cuff devices obtain a distance between the plurality of wireless cuff devices wirelessly, and wirelessly transmit information on the distance to the blood pressure pulse wave analyzing apparatus.

46. The blood pressure pulse wave analyzing system according to claim 43, further comprising:
a wireless sensor that is mounted on the subject at a position corresponding to a heart of the subject, detects a behavior of the heart, and wirelessly transmits heart behavior information, the behavior being a source of a pulse wave, wherein
the wireless sensor and each of the plurality of wireless cuff devices obtain a distance between the wireless sensor and each of the plurality of wireless cuff devices wirelessly, and wirelessly transmit information on the distance to the blood pressure pulse wave analyzing apparatus.

47. The blood pressure pulse wave analyzing system according to claim 29, wherein:
the blood pressure pulse wave analyzing apparatus is set as a first wireless device, and a wireless cuff device of the plurality of wireless cuff devices is set as a second wireless device, and transmission data of the second wireless device is synchronized with a reference clock of the first wireless device and transmitted;
the first wireless device acquires a time Ta that is a time from the reference clock to transmission of a first signal to the second wireless device and a time Tb that is a time from the transmission of the first signal to reception of a response signal from the second wireless device;
the first wireless device calculates a time Td that is a time from the reference clock to reception of the first signal by the second wireless device based on the time Ta and the time Tb;
the first wireless device transmits the time Td to the second wireless device in a next transmission cycle of the first signal; and
the second wireless device transmits data in a range corresponding to the time Td.

48. The blood pressure pulse wave analyzing system according to claim 47, wherein:
the second wireless device calculates timing of the reference clock of the first wireless device from the time Td; and
the second wireless device transmits data in a range corresponding to an interval of the reference clock.

49. The blood pressure pulse wave analyzing system according to claim 47, wherein:
the second wireless device includes an AD converter, a first memory, and a second memory, the AD converter performing AD conversion on transmission information, the first memory storing data after the AD conversion;
the second memory stores a position of a write pointer of the first memory when the first signal is received and the time Td indicated by the received first signal; and
the second wireless device controls, based on the stored position of the write pointer and the stored time Td, a range of data to be transmitted by setting a readout position from the first memory.

50. The blood pressure pulse wave analyzing system according to claim 49, wherein the second wireless device sets, as an end point of the readout position, AD conversion data that is earlier than the position of the write pointer stored in the second memory by a result of dividing the time Td by a sampling period of the AD converter.

51. The blood pressure pulse wave analyzing system according to claim 49, wherein
the AD converter is a ΔΣ type AD converter.

52. The blood pressure pulse wave analyzing system according to claim 51, wherein:
the ΔΣ type AD converter oversamples the transmission information; and
the second wireless device downsamples data after the oversampling.

53. The blood pressure pulse wave analyzing system according to claim 47, wherein:
when the first wireless device fails to receive the response signal from the second wireless device, the first wireless device transmits a retransmission signal to the second wireless device at a time Ta′ later than the time Ta;
the first wireless device further acquires the time Ta′ and a time Tb, the time Ta′ being a time from the reference clock to transmission of the retransmission signal to the second wireless device, the time Tb being a time from the transmission of the retransmission signal to the reception of the response signal from the second wireless device;
the first wireless device calculates, based on the time Ta′ and the time Tb, a time Td′ that is a time from the reference clock to the reception of the retransmission signal by the second wireless device;
the first wireless device transmits the time Td′ to the second wireless device in the next transmission cycle of the retransmission signal; and
the second wireless device transmits data in a range corresponding to the time Td′.

54. The blood pressure pulse wave analyzing system according to claim 47, wherein
when the plurality of wireless cuff devices are set as a plurality of second wireless devices,
the first wireless device acquires a plurality of times Ta that are times from the reference clock to the transmission of the first signal to the plurality of second wireless devices, respectively, and a plurality of times Tb that are times from the transmission of the first signal to the reception of the response signal from the plurality of second wireless devices, respectively,
the first wireless device calculates, based on the plurality of times Ta and the plurality of times Tb, a plurality of times Td that are times from the reference clock to the reception of the first signal by the plurality of second wireless devices, respectively, and
the first wireless device transmits a time Td corresponding to each of the plurality of second wireless devices to a corresponding one of the plurality of second wireless devices in the next transmission cycle of the first signal, the time Td being among the plurality of times Td, and
each of the plurality of second wireless devices transmits data in a range corresponding to the time Td corresponding to the own second wireless device, the time Td being among the plurality of times Td.

55. The blood pressure pulse wave analyzing system according to claim 49, wherein
the first memory is a ring buffer.

56. The blood pressure pulse wave analyzing system according to claim 49, wherein
the second memory is a FIFO memory.
